(19) United States
(12) Patent Application Publication
(10) Pub. No.: US 2022/0369973 A1
(43) Pub. Date: Nov. 24, 2022

(54) EXTENDED REALITY SYSTEM TO TREAT SUBJECTS ASSOCIATED WITH AUTISM SPECTRUM DISORDER

(71) Applicants: Genentech, Inc., South San Francisco, CA (US); Hoffmann-La Roche Inc., Little Falls, NJ (US)

(72) Inventors: Christopher Hughes CHATHAM, Little Falls, NJ (US); Mikael John Lars ELIASSON, San Anselmo, CA (US); Kevin SANDERS, Belmont, CA (US); Caitlin Anne PATTERSON, Berkeley, CA (US); Matteus Jiawei PAN, San Francisco, CA (US); Holly Anne UBER, South San Francisco, CA (US)

(21) Appl. No.: 17/845,960

(22) Filed: Jun. 21, 2022

Related U.S. Application Data
(63) Continuation of application No. PCT/US2020/065805, filed on Dec. 18, 2020.
(60) Provisional application No. 62/952,804, filed on Dec. 23, 2019.

(51) Int. Cl. A61B 5/16; A61B 5/00; G06F 3/01; G16H 20/70

(52) U.S. Cl. CPC A61B 5/163; A61B 5/4803; G06F 3/013; G16H 20/70

(57) ABSTRACT

Methods and systems for assessing autism spectrum disorder (ASD) are described herein. Data identifying one or more behaviors associated with ASD may be received. A scenario specifying a plurality of different tasks may be received. An extended reality (XR) device may present and XR environment to a subject. The XR device may present, in the XR environment, a first task of the plurality of different tasks. Based on a subject interaction with one or more objects in the XR environment, interaction data may be calculated. Based on the interaction data, at least one second task may be selected from the plurality of different tasks. The at least one second task may be configured to train a different skill as compared to the first task. The XR environment may be modified to present the second task.

### CROSS-REFERENCE TO RELATED APPLICATIONS

This application claims priority to U.S. Provisional Patent Application 62/952,804, titled “DIAGNOSTIC DEVICE, SYSTEM AND METHOD FOR MONITORING AND ASSESSING SUBJECTS ASSOCIATED WITH AUTISM SPECTRUM DISORDER” and filed Dec. 23, 2019, the disclosure of which is hereby incorporated by reference in its entirety.

### FIELD

Aspects described herein generally relate to medical treatment and medical devices for improved subject testing and analysis. More specifically, aspects described herein provide devices, systems and methods for addressing social, communication, and sensory limitations, such as those experienced by individuals experiencing Autism Spectrum Disorder, in a subject by active testing, passive monitoring, and/or treatment of the subject.

### BACKGROUND

Autism spectrum disorder (ASD) encompasses a broad range of conditions which may negatively impair an individual's social, communicative, and/or behavioral capabilities. Individuals experiencing ASD may experience difficulty communicating and interacting with others, may have particularly restricted interests, and/or may exhibit repetitive behaviors. For example, daily life tasks involving social interaction might present a particular difficulty for an individual experiencing ASD. ASD is often accompanied by sensory sensitivities and medical issues such as gastrointestinal disorders, seizures or sleep disorders, as well as mental health challenges such as anxiety, depression and attention issues. As such, individuals experiencing ASD may have difficulties at school, at work, and in other social contexts.

Various treatment methods exist for ASD. Generally, early identification of ASD symptoms in children is valuable, as early intervention strategies (e.g., therapy to help children experiencing ASD talk, walk, and generally otherwise interact with others) can be beneficial. Applied Behavior Analysis (ABA), a common approach, entails encouraging positive behaviors (e.g., social interaction) and discouraging negative behaviors (e.g., being withdrawn or non-communicative). Within the category of ABA, a number of approaches exist, including discrete trial training (e.g., testing and rewarding positive behavior in discrete tasks), early intensive behavioral intervention, pivotal response training (e.g., encouraging a subject to learn monitor their own behavior), verbal behavior intervention, occupational therapy (e.g., helping the subject have independently by learning to dress, eat, bathe, and perform other tasks), sensory integration therapy (e.g., helping the subject handle unwelcome sights, sounds, and smells), and the like. Other approaches include modifying the individual's diet, using medication, and the like.

The above methods have many drawbacks. Individuals experiencing ASD may withdraw from social interaction, meaning that they may be less likely to regularly seek therapy. Moreover, even if an individual experiencing ASD is willing to regularly participate in therapy, such therapy sessions might not be frequent enough to improve the individual's symptoms. It can also be difficult to moderate the severity of sensory inputs for an individual experiencing ASD, meaning that it may be difficult to gradually acclimate individuals to greater intensities of sensory input. For example, it may be difficult (and cost-prohibitive) to gradually introduce more and more complex social interactions in a therapeutic setting. Additionally, it can be extremely costly to have a clinician continually monitor the performance of a subject throughout a training process. For example, training for a subject with ASD can take hours, such that the cost of such training can be quite high if a clinician must continually monitor and modify aspects of the training.

### SUMMARY

The following presents a simplified summary of various aspects described herein. This summary is not an extensive overview, and is not intended to identify required or critical elements or to delineate the scope of the claims. The following summary merely presents some concepts in a simplified form as an introductory prelude to the more detailed description provided below.

To overcome limitations in the prior art described above, and to overcome other limitations that will be apparent upon reading and understanding the present specification, aspects described herein are directed towards measuring and ameliorating one or more symptoms of social, communicative, and/or sensory deficits in an individual exhibiting one or more such symptoms using an extended reality (e.g., an virtual, augmented, and/or mixed reality) environment.

A computing device may be coupled to (either indirectly through a data connection or directly and thus also physically be a part of) an extended reality (XR) device, which may be capable of providing an XR environment. The computing device may receive data identifying one or more behaviors associated with symptoms of ASD. The computing device may receive a scenario specifying a plurality of different tasks configured to train skills associated with the one or more behaviors associated with the symptoms of ASD. The computing device may cause the XR device to present an XR environment to a subject, and may cause the XR device to present, in the XR environment and to the subject, at least one first task of the plurality of different tasks specified in the scenario. That at least one first task may be configured to train a first skill associated with improvement of the one or more behaviors associated with the symptoms of ASD. The at least one first task may be configured to prompt the subject to interact with one or more objects in the XR environment. The computing device may detect a subject interaction with the one or more objects in the XR environment. The computing device may calculate, based on the one or more subject interactions, a behavioral score indicative of the one or more behaviors associated with the symptoms of ASD. The computing device may select, from the plurality of different tasks and based on the behavioral score indicative of the one or more behaviors associated with the symptoms of ASD, at least one second task of the plurality of different tasks. The second task may be different from the first task, and may be configured to train a second skill associated with improvement of the one or more behaviors associated with the symptoms of ASD.

Additionally and/or alternatively, existing data may be collected for a subject. The existing data may comprise information on an intake survey and/or clinical instrument filled out by a clinician, caregiver, and/or subject, electronic medical record data, and/or the presence/absence of one or more biomarkers. An extended reality environment may be provided, and therapeutic data may be collected using that environment. Such an extended reality environment may comprise video and/or audio and may comprise one or more scenarios, such as one or more of a testing scenario, an observational scenario, a learning scenario, a practical scenario, and/or a relaxation scenario. The therapeutic data may comprise data parameters such as implicit nonverbal communication parameters, nonverbal communication parameters, verbal communication parameters, sensory intensity parameters, sensory complexity parameters, sensory predictability parameters, social intensity parameters, and the like. The therapeutic data may be compared to the existing data, and the extended reality environment may be modified. For example, the intensity and/or degree of difficulty of various sensory challenges within the extended reality environment may be modified.

These and additional aspects will be appreciated with the benefit of the disclosures discussed in further detail below.

### BRIEF DESCRIPTION OF THE DRAWINGS

A more complete understanding of aspects described herein and the advantages thereof may be acquired by referring to the following description in consideration of the accompanying drawings, in which like reference numbers indicated like features, and wherein:

FIG. 1 depicts an illustrative computer system architecture that may be used in accordance with one or more illustrative aspects described herein.

FIG. 2 depicts an illustrative extended reality device.

FIG. 3 shows a flow chart comprising steps which may be performed, using an extended reality device, to monitor, assess, and train symptoms of ASD.

FIG. 4 depicts a diagram representing how different scenarios may be presented as part of the extended reality environment.

FIG. 5 shows a flow chart comprising steps which may be performed, using an extended reality device, to monitor, assess, and train symptoms of ASD.

FIG. 6A shows an example of an extended reality environment, showing an example where a subject is prompted to order food.

FIG. 6B shows an example of output associated with behaviors, by a subject, in an XR environment.

### DETAILED DESCRIPTION

In the following description of the various embodiments, reference is made to the accompanying drawings identified above and which form a part hereof, and in which is shown by way of illustration various embodiments in which aspects described herein may be practiced. It is to be understood that other embodiments may be utilized and structural and functional modifications may be made without departing from the scope described herein. Various aspects are capable of other embodiments and of being practiced or being carried out in various different ways.

As a general introduction to the subject matter described in more detail below, aspects described herein are directed towards assessing and/or treating one or more symptoms of social, communicative, and/or sensory deficits in an individual using an augmented reality. A method may include identifying a subject suffering from social, communication and/or sensory limitations. The method may further include determining at least one data parameter as a reference value for the subject. Such determining may be during a predefined time window and/or an intake session. The data parameter may, for example, include one or more parameters provided in or on an intake survey or clinical instrument filled out by a clinician or the subject; historical electronic medical record data; and/or digital biomarker data. The method may further include delivering a progressive set of therapeutic interventions to the subject through use of an extended reality apparatus (as used herein, unless explicitly stated otherwise, reference to any one of an extended, virtual, augmented, and/or mixed reality apparatus may also or alternative include the others). By comparing at least one newly determined value of the data parameter from the subject to the reference value of the data parameter, a difference may be calculated, which may indicate an improvement or worsening of symptoms. Such steps may be repeated until improvement in social and communication functions and/or daily living skills are obtained or other course of action is detected. The therapeutic extended reality interventions may include one or more phases selected from the group including: observational training scenarios, instrumental training scenarios, affective evaluation of performance, and virtual “cool down” rooms or safe spaces allowing psychological and physiological arousal to return to baseline. The method may further include defining observational training or instrumental training scenarios, staging of such scenarios, enabling clinicians and caregivers to interact with and/or monitor the subject in extended reality, incorporating all resulting data into a scoring system, and the like.

As another example, and as will also be described herein, a computing device may be coupled to an extended reality (XR) device. That XR device may be capable of providing one or more of a virtual, augmented, or mixed reality environment. The computing device may receive data identifying one or more behaviors associated with symptoms of ASD. For example, the one or more behaviors may include not looking a speech participant in the eye during a conversation. The computing device may receive a scenario specifying a plurality of different tasks. That plurality of different tasks may be configured to train skills associated with the one or more behaviors associated with the symptoms of ASD. For example, one of the plurality of different tasks may include training a subject to look a speech partner in the eyes. The computing device may cause the XR device to present an XR environment to a subject. The computing device may also cause the XR device to present, in the XR environment and to the subject at least one first task of the plurality of different tasks specified in the scenario. That at least one first task may be configured to train a first skill associated with improvement of the ne or more behaviors associated with the symptoms of ASD. For example, as indicated above, the at least one first task may include training a subject to look a speech partner in the eyes. The at least one first task may be configured to prompt the subject to interact with one or more objects in the XR environment. Such an interaction could include, for example, looking an avatar object the eyes, standing an appropriate distance away from the avatar object suing a virtual wallet to pay for goods at a store, or the like. The computing device may detect a subject interaction with the one or more objects in the XR environment. For example, the computing device may monitor the eye movement of a subject to generate eye tracking data, then process the eye tracking data to determine whether or not the subject is looking at an avatar object. The computing device may calculate, based on the one or more subject interactions, a behavioral score indicative of the one or more behaviors associated with the symptoms of ASD. For example, the computing device may provide a score as to how well a subject looked into the eyes of a speech partner. The computing device may select, from the plurality of different tasks and based on the behavioral score indicative of the one or more behaviors associated with the symptoms of ASD, at least one second task of the plurality of different tasks. The second task may be different from the first task, and may be configured to train a second skill associated with the improvement of one or more behaviors associated with the symptoms of ASD. For example, the second task may require that a subject vocally interact with an avatar object. The computing device may modify, based on the scenario, the XR environment to present the second task.

It is to be understood that the phraseology and terminology used herein are for the purpose of description and should not be regarded as limiting. Rather, the phrases and terms used herein are to be given their broadest interpretation and meaning. The use of “including” and “comprising” and variations thereof is meant to encompass the items listed thereafter and equivalents thereof as well as additional items and equivalents thereof. The use of the terms “connected,” “coupled,” “engaged” and similar terms, is meant to include both direct and indirect connecting, coupling, and engaging.

### Extended Reality Environment and Computing Devices

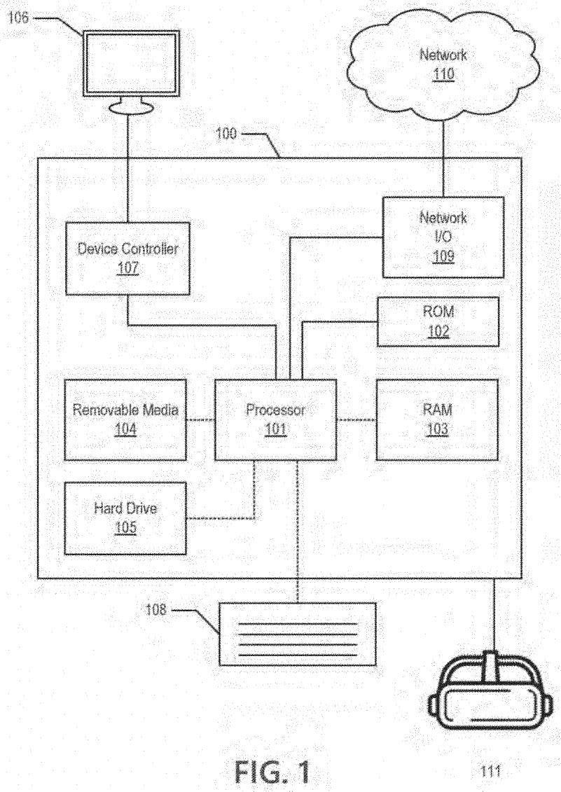

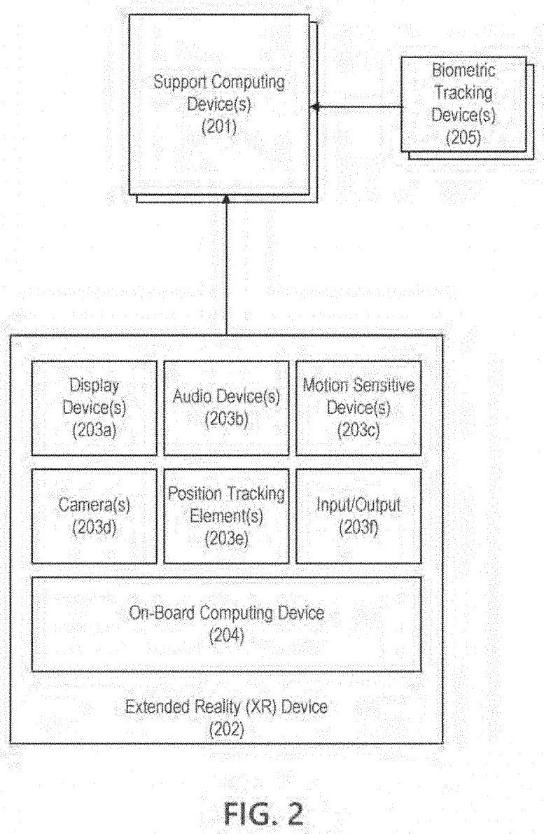

FIG. 1 is a block diagram showing hardware elements of an example computing device 100. As will be explained in greater detail below, such a computing device may be used to provide an extended reality environment. For example, a computing device such as the example computing device 100 may include one or more processors that execute instructions (stored in a memory) that cause the computing device to provide an extended reality (e.g., a virtual, augmented, and/or mixed reality) environment using an extended reality device 111. A computing device, such as the example computing device 160, may be part of and/or communicatively connected to an extended reality device 111. The extended reality device 111 may be configured to provide an extended (e.g., one or more of a virtual augmented and/or mixed reality) environment to a subject. The extended reality device 111 may, as explained in greater detail below with respect to FIG. 2, use the computing device 100 for one or more portions of providing the augmented reality environment to the subject. In some examples, a computing device such as is described herein may omit one or more of the elements shown in FIG. 1.

The computing device 100 may include one or more processors 101, which may execute instructions of a computer program to perform any of the features described herein. The instructions may be stored in a computer-readable medium or memory, to configure the operation of the processor 101. For example, instructions may be stored in a read-only memory (ROM) 102, a random access memory (RAM) 103, a removable media 104, such as a Universal Serial Bus (USB) drive, compact disk (CD) or digital versatile disk (“DVD”), a floppy disk drive, cloud or network storage, or another storage medium. Instructions may also be stored in an attached (or internal) hard drive 105. The computing device 100 may include one or more output devices, such as a display 106 (e.g., an external television or monitor), and may include one or more output device controllers 107, such as a video processor. There may also be one or more user input devices 108, such as a remote control, keyboard, mouse, touch screen, microphone, camera (e.g., to capture input for user gestures), etc. The computing device 100 may also include one or more network interfaces, such as a network input/output (I/O) circuit 109 (e.g., a network card) to communicate with an external network 110. The network input/output circuit 109 may be a wired interface, wireless interface, or a combination of the two. Additionally, the device may include a location-detecting device, such as a global positioning system (GPS) microprocessor 111, which can be configured to receive and process global positioning signals and calculate, with possible assistance from an external server and antenna, a geographic position of the device.

While FIG. 1 illustrates an example of a hardware configuration that may be used in some arrangements, in other arrangements, one or more components may be implemented as software. In addition, modifications may be made to add, remove, combine, divide, etc. components of the computing device 100. Additionally, the components may be implemented using basic computing device 100. Additionally, the components may be implemented using basic computing devices and components, and the same components (e.g., processor 101, ROM storage 102, display 106, etc.) may be used to implement any of the other computing devices and components described herein. For example, one or more of the various components described herein may be implemented using computing devices having components such as a processor executing computer-executable instructions stored on a computer-readable medium, or shown in FIG. 1. Some or all of the entities described herein may be software based, and may co-exist in a common physical platform (e.g., a requesting entity can be a separate software process and program from a dependent entity, both of which may be executed as software on a common computing device).

FIG. 2 depicts an example of an extended reality (XR) device 202, which may be the same or similar as the extended reality device 111. The XR device 202 may be communicatively connected to an on-board computing device 204, which may be the same or similar as the computing device 100. The XR device 202 may include a plurality of different elements such as display devices 203a, audio devices 203b, motion sensitive devices 203c, cameras 203d, position tracking elements 203e, and input/output devices(s) 203f. Such elements may additionally and/or alternatively be referred to as sensors. Other such elements, not shown, may include in-ear electroencephalographic (EEG) and/or heart rate variability (HRV) measuring devices, scalp and/or forehead-based EEG and/or HRV measurement devices, eye-tracking devices (e.g., using infrared), or the like. The XR device 202 may further include a support computing devices(s) 201, which may be the same or similar as the computing device 100. Not all elements shown in FIG. 2 need to be present for operation of the XR device 202. For example, the XR device 202 might not have the cameras 203d. As another example, the XR device 202 might lack any of the support computing device(s) 201, such that the on-board computing device 204 directly interfaces with the display devices 203a, the audio devices 203b, the motion sensitive devices 203c, the cameras 203d, the position tracking elements 203e, and/or the input/output 203f to provide an extended reality environment. As yet another example, the support computing devices(s) 201 may be sufficiently powerful enough such that the on-board computing device 204 may be omitted. The support computing device(s) may be communicatively coupled (e.g., over a network, such as the network 110) to biometric tracking device(s) 205 and database(s) 206.

In some instances, the on-board computing device 204 and/or the support computing device(s) 201 might not have any particular processing power or functionality to provide an extended reality environment. The on-board computing device 204 and/or the support computing device(s) 201 may include, for example, relatively underpowered processors which provide rudimentary video and/or audio. In other instances, the on-board computing device 204 and/or the support computing device(s) 201 may, for example, include relatively powerful processors which provide highly realistic video and/or audio.

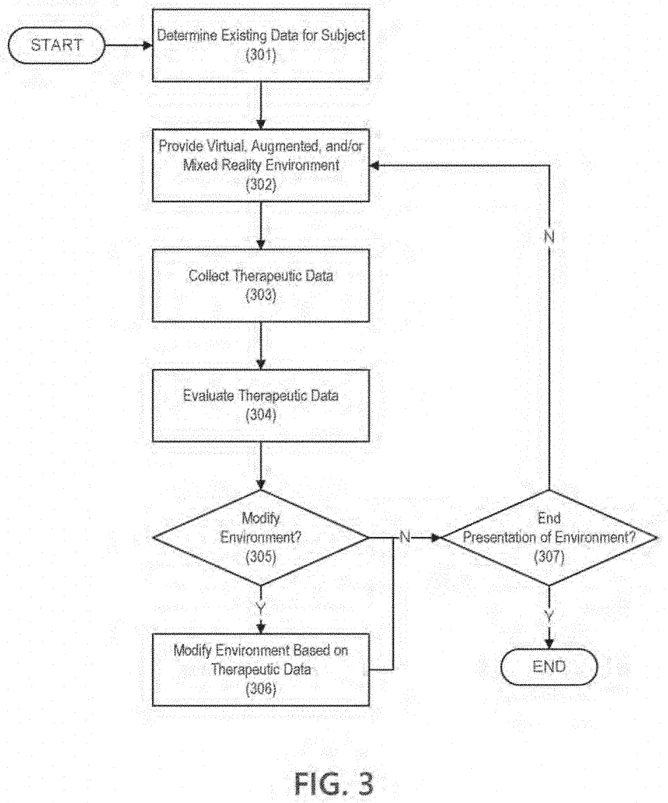

The XR device 202 may provide an extended reality (e.g., a virtual, augmented, and/or mixed reality) environment to a user, such as a subject. In general, virtual reality environments provide an entirely virtual world, whereas augmented reality and/or mixed reality environments mix elements in the real world and the virtual world. The XR device 202 may be a device specifically configured to provide an extended reality environment (e.g., an extended reality headset), or may be a combination of devices (e.g., a smartphone inserted into and/or communicatively coupled to a headset) which, when operated in a particular manner, provides an extended reality environment. The XR device 202 may be said to be untethered at least in part because it may lack a physical connection to another device (and, e.g., may be battery powered). If the XR device 202 is connected to another device (e.g., the support computing device(s) 201, a power source, or the like), it may be said to be tethered. Examples of the XR device 202 may include the VALVE INDEX virtual reality device developed by Valve Corporation of Bellevue, Wash., the OCULUS QUEST virtual reality device sold by Facebook Technologies, LLC of Menlo Park, Calif., and the HTC VIVE virtual reality device sold be HTC Corporation of New Taipei City, Taiwan. Examples of the XR device 202 may also include smartphones which may be placed into a headset for extended reality purposes, such as the GEAR VR product sold by Samsung Group of Seoul, South Korea. Examples of the XR device 202 may also include the augmented reality headsets sold by magic Leap, Inc. of Plantation, Fla., the HOLOLENS mixed reality headsets sold by Microsoft Corporation of Redmond, Wash., and NREAL LIGHT headsets sold by Hangzhou Tainio Technology Co., Ltd. of Beijing, China, among others. Examples of the XR device 202 may also include audio-based devices, such as the ECHO FRAMES sold by Amazon, Inc of Seattle, Wash. All such extended reality devices may have different specifications. For example, some extended reality devices may have cameras, whereas others might not. These are merely examples, and other AR/VR systems may also or alternatively be used. Moreover, as will be described in further detail below (and, e.g., with respect to the steps shown in FIG. 3), either or both the on-board computing device 204 and/or the support computing device(s) 201 may perform the steps described herein. Accordingly, the disclosure herein may be performed exclusively by the on-board computing device 204 (e.g., such that the XR device 202 is untethered), by the support computing device(s) 201 (e.g., that the XR device 202 is tethered to a computing device, such as in a laboratory setting), and/or a combination thereof (e.g., such that the on-board computing device 204 performs some steps described herein, the support computing device(s) 201 perform other steps described herein, and the devices collectively perform all steps described herein).

The support computing device(s) 201 may provide all or portions of an extended reality environment to the XR device 202, e.g., as used by a tethered OCULUS RIFT. For example, the support computing device(s) 201 may provide a video data stream to the XR device 202 that, when displayed by the XR device 202 (e.g., through the display devices 203a), shows a virtual world. Such a configuration may be advantageous where the XR device 202 (e.g., the on-board computing device 204 that is part of the XR device 202) is not powerful enough to display a full extended reality environment. The support computing device(s) 201 need not be present for the XR device 202 to provide an extended reality, augmented reality, and/or mixed reality environment. For example, where the on-board computing device 204 is sufficiently powerful, the support computing device(s) 201 may be omitted (or, alternatively, to be considered to have been implemented within on-board computing device 204), e.g., an untethered OCULUS QUEST.

The display devices 203a may be any devices configured to display all or portions of an extended reality environment. Such display devices 203a may include, for example, flat panel displays, such as one or more liquid-crystal display (LCD) panels. The display devices 203a may be the same or similar as the display 106. The display devices 203a may be singular or plural, and may be configured to display different images to different eyes of a user. For example, the display devices 203a may include one or more display devices coupled with lenses (e.g., Fresnel lenses) which separate all or portions of the displays for viewing by different eyes of a user.

The audio devices 203b may be any devices which may receive and/or output audio associated with an extended reality environment. For example, the audio devices 203b may include speakers which direct audio towards the ears of a user. As another example, the audio devices 203b may include one or more microphones which receive voice input from a user. The audio devices 203b may be used to provide an audio-based extended reality environment to a user of the XR device 202.

The motion sensitive devices 203c may be any elements which receive input related to the motion of a user of the XR device 202. For example, the motion sensitive devices 203c may include one or more accelerometers which may detect when a user of the extended reality device is moving (e.g., leaning, moving forward, moving backwards, turning, or the like). Three dimensional accelerometers and/or gyroscopes may be used to detect full motion of the XR device 202. Optional external facing cameras 203d may be used for 3D orientation as well. The motion sensitive devices 203c may permit the XR device 202 to present an extended reality environment which changes based on the motion of a user.

The cameras 203d may be used to aid in the safety of the user as well as the presentation of an extended reality environment. The cameras 203d may be used to monitor the surroundings of a user so as to avoid the user inadvertently contacting elements (e.g., walls) in the real world. The cameras 203d may additionally and/or alternatively monitor the user (e.g. the eyes of the user, the focus of the user's eyes, the pupil dilation of the user, or the like) to determine which elements of an extended reality environment to render, the movement of the user in such an environment, or the like.

The position tracking elements 203e may be any elements configured to aid in the tracking of the position and/or movement of the XR device 202. The position tracking elements 203e may be all or portions of a system of infrared emitters which, when monitored by a sensor, indicate the position of the XR device 202 (e.g., the position of the XR device 202 in a room). The position tracking elements 203e may be configured to permit “inside-out” tracking, where the XR device 202 tracks the position of one or more elements (e.g., the XR device 202 itself, a user's hands, external controllers, or the like) or “outside-in” tracking, where external devices and in tracking the position of the one or more elements. The position tracking elements 203e may aid in determining a position of a user in both the real world (e.g., in a room) and in an extended reality environment. For example, a user might be simultaneously a distance from a real-world object (e.g., a wall) and an extended reality object (e.g., an avatar object).

The input/output 203f may be configured to receive and transmit data associated with an extended reality environment. For example, the input/output 203f may be configured to communicate data associated with movement of a user to the support computing device(s) 201. As another example, the input/output 203f may be configured to receive information associated with other users of a massively multiplayer extended reality environment.

The on-board computing device 204 may be configured to provide, via the display devices 203a, the audio devices 203b, the motion sensitive devices 203c, the cameras 203d, the position tracking elements 203e, and/or the input/output 203f, the extended reality environment. The on-board computing device 204 may include one or more processors (e.g., a graphics processor), storage (e.g., that stores extended reality programs), or the like. In one or more arrangements, the on-board computing device 204 may be powerful enough to provide the extended reality environment without using the support computing device(s) 201, such that the support computing device(s) 201 might not be required and might not be connected to the XR device 202. In other configuration, the support computing device(s) 201 and the on-board computing device 204 may work in tandem to provide the extended reality environment. In other configurations, the XR device 202 might not have the on-board computing device 204, such that the support computing device(s) 201 interface with the display devices 203a, the audio devices 203b, the motion sensitive devices 203c, the cameras 203d, the position tracking elements 203e, and/or the input/output 203f directly.

The above-identified elements of the XR device 202 are merely examples. The XR device 202 may have more or similar elements. For example, the XR device 202 may include in-ear EEG and/or HRV measuring devices, scalp and/or forehead-based EEG and/or HRV measurement devices, eye-tracking devices (e.g., using cameras directed at users' eyes, pupil tracking, infrared), or the like.

### ASD Assessment and Treatment in an Extended Reality Environment

Discussion will now turn to use of virtual, augmented, and/or mixed reality to monitor, assess, and/or ameliorate symptoms of ASD.

FIG. 3 shows a flow chart comprising steps which may be performed, using an extended reality device such as the XR device 202, to monitor, assess, and treat symptoms of ASD. The steps shown in FIG. 3 may be performed by a computing device, such as the computing device 100, the on-board computing device 204, and/or the support computing device(s) 201, and may be all or portions of an algorithm. The steps shown in FIG. 3 may be performed to monitor, assess, and/or treat skills associated with one or more symptoms of social, communicative, and/or sensory deficits in a tested subject.

In step 301, the on-board computing device 204 and/or the support computing device(s) 201 may collect existing data associated with a subject. The existing data may be received via a database, such as from the database(s) 206. The existing data may include personal data about the subject. The existing data may include information provided via an intake survey and/or clinical instrument filled out by one or more of a clinician, a caregiver, and the subject. The existing data may include electronic medical record data. The existing data may include any other similar forms of historical information about a subject, and need not originate in a clinical setting. For example, the existing data may include information reflecting feedback from a parent or spouse of the subject. The existing data associated with the subject may be used (e.g., by XR device 202) to calculate a baseline of a subject. The baseline may be associated with a period of time, such as a previous therapeutic session. The on-board computing device 204 and/or the support computing device(s) 201 may use the existing data to calculate the status quo or baseline conditions of a subject.

The existing data may include information indicating the presence and/or absence of one or more biomarkers regarding the presence, absence, and/or severity of ASD. For example, some differences in the gastrointestinal system of a subject may indicate the presence of ASD. As another example, certain differences in the immunological system, neurological system, and/or toxicological system of a subject may indicate the presence of ASD. Digital biomarkers from computerized intake systems may be used as well.

Determining the existing data associated with a subject may include receiving the existing data from one or more external sources, such as the database(s) 206. The on-board computing device 204 and/or the support computing device(s) 201 may store the existing data, and determining the existing data may include retrieving the existing data from those computing devices. For example, determining the external data may include retrieving, from an external database (e.g., the database(s) 206), one or more portions of the external data. Determining the existing data associated with the subject may additionally and/or alternatively include receiving input from a subject. For example, determining the external data may include retrieving, via a user interface and from a caregiver, a clinician, and/or the subject, the external data.

In step 302, the XR device 202 may provide an extended reality environment (XR environment). The extended reality environment may be configured to determine one or more data parameters, as detailed below. The extended reality environment may be presented as part of one or more therapeutic interventions (e.g., a series of tasks) on a subject. For example, the extended reality environment may be presented as part of a therapeutic intervention and/or a series of therapeutic interventions. As a particular example, the extended reality environment may be configured to monitor, assess, and/or train skills associated with at least one symptom associated with ASD (e.g., social communication and social interaction difficulties, restricted, repetitive patterns of behavior, interests or activities, or the like).

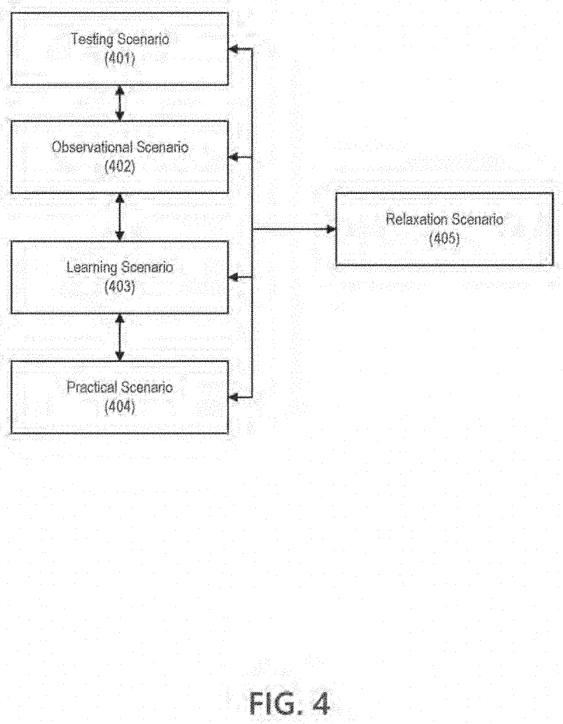

The extended reality environment (which may, e.g., be provided by XR device 202 at step 302) may present a user with and/or otherwise include one or more tasks (which may be part of a scenario, which might include one or more tasks). The one or more tasks may include one or more observational tasks, wherein one or more social, communication and/or memory skills are demonstrated to the subject. Additionally and/or alternatively, the extended reality environment may present one or more tasks, such as daily living skill tasks (e.g., interacting with bank tellers, waiters/waitresses, going through security at the airport), personal hygiene tasks, or the like. The one or more tasks may include one or more learning tasks, wherein the subject is caused to demonstrate their learning of one or more social, communication and/or sensory skills by means of interacting with objects and/or avatars in the extended reality environment. The one or more tasks may include one or more practical tasks in which the subject may learn to schedule, plan, evaluate and/or recall one or more learned social, communication and/or sensory skills in the world outside of the extended reality environment. The one or more tasks may include one or more, calming tasks in which the subject may escape one or more of the observational, learning and practical tasks while still using an extended reality device. For example, the one or more calming tasks may include meditation, a personal area associated with the subject, or the like. All such tasks may be associated with training material (e.g., brochures, smartphone apps, etc.) which help educate the subjects as to the one or more social, communication and/or sensory skills that are tested in the extended reality environment. The one or more calming tasks may be interleaved with other tasks so as to allow the subject to return to a baseline and/or to permit the subject to rest recuperate from other tasks, which may in aggregate overwhelm the subject. Such tasks are described in more detail in FIG. 4, which is discussed further below.

As an example, the one or more tasks may include presenting, via an XR environment presented using the XR device 202 and to the subject, one or more welcoming or unwelcoming groups of avatar objects, where welcoming or unwelcoming behavior is evidenced through verbal and nonverbal behavior including eye gaze, physical proximity, skin tone, pupil dilation, posture, arm position, physical orientation towards versus away from the subject, mutual eye gaze, width of the eye sockets by contraction of the Orbicularis oculi or frontalis muscle, and the speed, pitch, prosody and semantic content of verbal utterances. As another example, the one or more tasks may include presenting, via an XR environment presented using the XR device 202 and to the subject, one or more avatar objects whose behavior with respect to one another and to the subject is characterized by time lagged verbal and nonverbal mimicry between the avatars, in terms of eye gaze, physical proximity, skin tone, pupil dilation, posture, arm position, physical orientation towards versus away from the subject, mutual eye gaze, width of the eye sockets by contraction of the Orbicularis oculi or frontalis muscle, and the speed, pitch, prosody and semantic content of verbal utterances.

The extended reality environment may be managed by a computing device, such as the support computing device(s) 201, external to an extended reality device. For example, the extended reality environment may be presented by an extended reality device such as the XR device 202, but control of that environment (e.g., deciding which scenarios to present deciding which sensory input to emulate) may be controlled by a separate computing device, such as an application executing on a tablet managed by a clinician. The clinician could control the environment in a variety of contexts, such as, e.g., when monitoring activity of the subject in an extended reality environment.

The extended reality environment may be augmented using video, text, or other learning material. For example, providing the extended reality environment may include the XR device 202 initially providing the subject with instructions (e.g., in the environment or elsewhere, such as on a tablet, in a brochure, etc.) indicating goals for the environment. In this manner, the subject may be informed of the one or more social, communicative, and/or sensory skills to be tested in the extended reality environment.

In step 303, the on-board computing device 204 and/or the support computing device(s) 201 may collect therapeutic data including one or more data parameters. The extended reality environment presented by the XR device 202 may be configured to collect (e.g., periodically) one or more data parameters associated with the subject in various scenarios (e.g., and performing one or more tasks). As a simple example, the extended reality environment may include a scenario emulating a virtual store, where the subject is tasked with using verbal communication skills to purchase an item, and where the subject's speech volume is monitored to ascertain if the subject speaks at an appropriate volume. The scenario may, but need not, emulate real-world circumstances wherein a subject experiencing ASD may experience difficulties. For example, the scenario may emulate a crowded party, presenting a circumstance wherein a subject experiencing ASD may have particular difficulty, and the subject's ability to ignore sudden distractions may be monitored. The scenario may test one or more such parameters. For example, a scenario may be configured to test both a subject's ability to speak with a stranger as well as the subject's ability to ignore distractions (e.g., unexpected loud noises).

The data parameters may include an information associated with the subject, the extended reality environment, the scenario, or the like. The data parameters may correspond to biomarker data received from, e.g., the biometric tracking device(s) 205, such that the digital parameters may include, e.g., the heart rate of a subject. The digital parameters need not come from sensors or devices associated with the extended reality device. For example, biomarker data may be collected using the extended reality device and/or external devices, such as the biometric tracking device(s) 205 (which may be, e.g., a subject's fitness device or other wearable device or monitor). As another example, heart rate data may be collected from the biometric tracking device(s) 205 (e.g., a subject's smart watch or smartphone). As yet another example, such heart rate data may come from an electroencephalogram (EEG) (which may be one of the biometric tracking device(s) 205). As yet another example, the digital parameters may include heart rate data superimposed on brain rhythm data as acquired using an EEG (which may be part of the biometric tracking device(s) 205).

The data parameters may be subjective or objective. For example, some data parameters may relate to a subjective evaluation of a subject's word use, whereas others may correspond to the objective measurement of the pupil dilation of the subject. The data parameters may include clinician-derived scores of the subject's social, communicative, and/or sensory functions. Additionally and/or alternatively, such data parameters may include clinician-derived scores of the subject's daily living skills. Such clinician-derived scores may be subjective or objective. such clinician-derived scores may originate from a device, such as a laptop or tablet, operated by the clinician. Similarly, the data parameters may include caregiver-derived scores of the subject's social, communicative, and/or sensory functions. Additionally and/or alternatively, such data parameters may include caregiver-derived scores of the subject's daily living skills. Such caregiver-derived scores may be subjective or objective. Such caregiver-derived scores may originate from a device, such as a smartphone, operated by the caregiver.

During the presentation of a scenario in an extended reality environment the on-board computing device 204 and/or the support computing device(s) 201 may collect data parameters including a subject's physical proximity to other individuals (e.g., avatars representing individual(s). Individuals experiencing ASD may tend to keep a too-wide or too-narrow distance between themselves and others. As such, the subject's physical proximity to individuals (e.g., avatars displayed in the scenario, whether or not such avatars represent real individuals or not) may be collected as one such data parameter. Such a data parameter may be collected using software providing the extended reality environment, as executing on a computing device such as the on-board computing device 204 and/or the support computing device(s) 201, and using sensors such as the motion sensitive devices 203c and/or the position tracking elements 203e. The physical proximity may indicate whether a subject in too close or too far from an individual (e.g., an avatar). As such, the collected data parameters indicating the subject's physical proximity to other individuals may indicate whether the data parameters satisfy a threshold associated with standing too closely and/or a threshold associated with standing too far away. Such thresholds might be modified to account for, for example, cultural differences, contextual differences, and the like. For example, a crowded party might have different thresholds as compared to an airport, and a socially appropriate distance in France might be slightly different than a socially appropriate distance in China. In this manner, the subject might be evaluated on whether, during interactions, the subject stands an acceptable distance from others.

During the presentation of a scenario in an extended reality environment, the on-board computing device 204 and/or the support computing device(s) 201 may collect data parameters including implicit nonverbal communication parameters, such as the pupil dilation and/or physical posutre of a subject. Such nonverbal communication parameters may indicate the feelings of a subject, including whether the subject is stressed (and/or undergoing a so-called “fight or flight” response). For example, a subject under stress may exhibit pupil dilation. The monitoring of such data parameters may be performed by an extended reality device, such as the XR device 202, and using sensors such as the motion sensitive devices 203c, the cameras 203d, and/or the position tracking elements 203e. As a particular example, one or more cameras of the cameras 203d may be configured to monitor the pupil size of a subject.

During the presentation of a scenario in an extended reality environment, the on-board computing device 204 and/or the support computing device(s) 201 may collect data parameters including nonverbal communication parameters, such as head nodding, whether the subject gazes at or away from another human and/or an avatar object, whether the user gazes at or away from the face of another human and/or an avatar object, whether the user gazes at or away from the eyes of another human and/or an avatar object, or the like. Such data parameters may be collected during participation of the subject in one or more tasks of a scenario, such as when prompted to look in particular directions. A subject experiencing ASD may be less likely to look at someone they are speaking to. In particular, because looking at someone else in the eyes may make the subject feel vulnerable, the likelihood that they look an avatar in the eyes may evince their comfort in a particular scenario and/or when performing a particular task. The monitoring of such data parameters may be performed by an extended reality device, such as the XR device 202, and using sensors such as the motion sensitive devices 203c, the cameras 203d, and/or the position tracking elements 203e.

During the presentation of a scenario in an extended reality environment by the XR device 202, the on-board computing device 204 and/or the support computing device(s) 201 may collect data parameters including verbal communication parameters, such as the speech speed, speech volume, speech fundamental frequency, speech shimmer, speech jitter, and speech frequency trajectory of a subject. Such data parameters may be collected during participation of the subject in one or more tasks of a scenario, such as when prompted to speak with an avatar. Such data parameters may indicated symptoms of ASD, as a subject experiencing ASD may, particularly if nervous, speak undesirably quickly, quietly, and/or modulate their voice in an unusual manner. Additionally, the scenario may test data parameters including verbal communication parameters which require analysis of the speech of the subject. Such analysis may render information regarding, for example, the directness and empathy of the subject. For example, the data parameters may include the speech vectorized word embedding of a subject. As another example, the data parameters may reflect semantic analysis of the speech of a subject, including a sentiment analysis of such speech. The monitoring of such data parameters may be performed via an extended reality device, such as the XR device 202, using sensors such as the audio devices 203b, and while the subject is in a scenario that comprises one or more tasks. Analysis of the speech of the subject may be performed during or after the scenario has been provided. For example, the speech of a subject may be recorded in text form (e.g., using a speech recognition algorithm), and then subsequently analyzed to evaluate word choice.

During the presentation of a scenario in an extended reality environment by the XR device 202, the on-board computing device 204 and/or the support computing device(s) 201 may collect data parameters including social intensity parameters, such as the presence of real or simulated other subjects, the number of such real or simulated subjects, the temporal coordination of those real or simulated subjects with respect to one another and with respect to the subject, the number of real or simulated other subjects which are engaged in joint as opposed to solitary activities, the number of subjects participating in each such joint activity, the relative rewards for social versus non-social activities, and/or the presence of shared versus unshared social goals with and among the real or simulated others. Such data parameters may be collected during participation of the subject in one or more tasks of a scenario, such as when prompted to walk across a simulated busy street or walk in a simulated crowded store. Such data parameters may indicate the overall difficulty of the scenario presented to the subject. For example, a crowded party with loud noises might present a particularly difficult scenario for a subject, whereas a quiet room with a single virtual avatar might present a relatively less difficult scenario.

During the presentation of a scenario in an extended reality environment by the XR device 202, the on-board computing device 204 and/or the support computing device(s) 201 may collect data parameters including sensory intensity parameters, such as auditory and spatial frequency content and auditory and spatial power at various frequencies. Such data parameters may be collected during participation of the subject in one or more tasks of a scenario, such as when prompted to perform tasks in a loud or otherwise obnoxious environment. Such data parameters may indicate the strength of sensory information received by a subject (e.g., how loud noises around the subject in the scenario are), which may, like the above, indicate an overall difficulty of the scenario for an individual experiencing ASD. The monitoring of such data parameters may additionally and/or alternatively be performed by an extended reality device, such as the XR device 202, and using sensors such as the audio devices 203b.

During the presentation of a scenario in an extended reality environment by the XR device 202, the on-board computing device 204 and/or the support computing device(s) 201 may collect data parameters including sensory complexity parameters, such as the number of auditory and visual objects in the virtual scenario. As with the above, the quantity of distracting and/or unwelcome elements in a scenario (e.g., the number of bright lights in a particular scenario) may affect the difficulty of such a scenario for an individual experiencing ASD. Also, the number of people (whether avatars or real people) in a scenario may affect the difficulty of such a scenario for an individual experiencing ASD.

During the presentation of a scenario in an extended reality environment by the XR device 202, the on-board computing device 204 and/or the support computing device(s) 201 may collect data parameters including sensory predictability parameters, such as determining a time series entropy of sensory intensity and complexity. Such data parameters may be collected during participation of the subject in one or more tasks of a scenario, such as when prompted to perform tasks in a crisis-like environment where sudden and unexpected problems occur. Individuals experiencing ASD may have a more difficult time dealing with sensory input which is unpredictable, such as sudden loud noises. As such, the data parameters may, like some of the other data parameters discussed above, evince the overall difficulty, for the subject of the scenario. Such data parameters may be collected using software providing the scenario in the extended reality environment, as executing on a computing device such as the on-board computing device 204 and/or the support computing device(s) 201.

The on-board computing device 204 and/or the support computing device(s) 201 may collect and evaluate all data parameters based on time and/or conditions of the scenario, including comparing first data parameters to second data parameters For one of more of the data parameters, the on-board computing device 204 and/or the support computing device(s) 201 may calculate a time lag. For example, the time lag of the maximum inter-agent correlation and/or the maximum inter-agent correlation may be calculated. The on-board computing device 204 and/or the support computing device(s) 201 may normalize one or more of the data parameters based on the conditions of the scenario. For example, the speech volume of a subject may be considered normal, even if high, if the scenario is also loud. The data parameters may include a weighted sum of values (e.g., of one or more of the above data parameters) under suitable linear or non-linear transformations. For instance, the data parameters may include a weighted sum of caregiver-reported, clinician-reported, and self-reported outcomes under linear or non-linear transformations. The data parameters may be for a particular time window and/or session. The on-board computing device 204 and/or the support computing device(s) 201 may collect data parameters at predetermined intervals, and such predetermined intervals may be based on, e.g., the number of data parameters collected, the intensity of the scenario, the fidelity desired in the data parameters, or the like.

In step 304, the on-board computing device 204 and/or the support computing device(s) 201 may evaluate the therapeutic data (including the one or more data parameters). The therapeutic data may be compared to the existing data discussed with respect to step 301. Step 304 may include the on-board computing device 204 and/or the support computing device(s) 204 determining whether the subject has improved or regressed with respect to symptoms of ASD.

Determining whether the subject has improved, remained static in their skill attainment, or regressed may include the on-board computing device 204 and/or the support computing device(s) 201 comparing the difference between the therapeutic data and the existing data with a predetermined threshold. The predetermined threshold may be set to avoid minor measurement variation from incorrectly indicating improvement or regression. That said, the therapeutic data need not necessarily show improvement in one or more dimensions to evince improvement by a subject. For example, a scenario presented in the virtual, augmented, and/or mixed environment presented in step 302 may be more complex (e.g., involve more avatars, involve more unexpected sensory information) than previous scenarios, such that a subject may perform worse in the scenario but may nonetheless improve relative to past behavior. Thus, as part of step 304, the on-board computing device 204 and/or the support computing device(s) 201 may normalize certain parameters (e.g., the implicit nonverbal communication parameters, the nonverbal communication parameters, the verbal communication parameters, and the like) based on information about the scenario provided (e.g., the sensory intensity parameters, the sensory complexity parameters, the sensory predictability parameters, the social intensity parameters, and the like).

In step 305, based on the evaluation, the on-board computing device 204 and/or the support computing device(s) 201 may determine whether to modify the scenario presented in the extended reality environment. Particularly, based on the evaluation of the therapeutic data (e.g., whether the subject is improving or regressing with respect to one or more skills), it may be desirable to modify the extended reality environment to encourage the subject to continue improving the one or more skills in, e.g., a different scenario. It may be advantageous to increase the difficulty of scenarios, particularly when a subject has developed a proficiency in easier scenarios. As an example, it may be beneficial, after determining that a subject has mastered simple social scenarios (e.g., eating dinner with a friend), for the on-board computing device 204 and/or the support computing device(s) 201 to modify the extended reality environment to present relatively more complicated social scenarios (e.g., a crowded party with strangers). If the answer to step 305 is yes, the flow chart proceeds to step 306. Otherwise, the flow chart proceeds to step 307.

In step 306, based on determining to modify the extended reality environment (e.g., to modify one or more scenarios presented in the extended reality environment), the on-board computing device 204 and/or the support computing device(s) 201 may cause the environment presented by the XR device to be modified based on the therapeutic data. The on-board computing device 204 and/or the support computing device(s) 201 may increase or decrease the intensity of sensory input of the extended reality environment by, for example, modifying an existing scenario or presenting a new scenario. The on-board computing device 204 and/or the support computing device(s) 201 may increase or decrease the complexity of the extended reality environment by, for example, modifying an existing scenario or presenting a new scenario. The context (e.g., location, identity of avatars, or the like) of the extended reality environment may be changed by the on-board computing device 204 and/or the support computing device(s) 201.

Modification of the extended reality environment by the on-board computing device 204 and/or the support computing device(s) 201 may include modifying the intensity or degree of difficulty of various sensory challenges within one, or more scenarios, such as modifying visual and/or auditory and/or tactile components such the frequency content (e.g., in the tactile, spatial and auditory frequency spectra), the intensity (e.g., amplitude and/or power in each frequency), the complexity (e.g., the number of visual objects, and/or distinct audio or tactile sources within the environment) and/or predictability (e.g., the time-series entropy) of these sensory features across time.

Modification of the extended reality environment by the on-board computing device 204 and/or the support computing device(s) 201 may include modifying the intensity or degree of difficulty of various social challenges within one or more scenarios, such as the presence of real or simulated other subjects, the number of these other real or simulated subjects, the temporal coordination of those subjects' behavior with respect to one another and with respect to the subject, the number of real or simulated other subjects which are engaged in joint as opposed to solitary activities, the number of subjects participating in each such joint activity, the relative rewards for social versus non-social activities, the presence of shared versus unshared social goals with and among the real or simulated others, the predictability of these social features across time, and the like.

Modification of the extended reality environment by the on-board computing device 204 and/or the support computing device(s) 201 may include modifying the intensity or degree of difficulty of various communication challenges of one or more scenarios, such as the valence and intensity of nonverbal behavior (e.g., body language, facial expressions, and physical proximity) between real and/or simulated users, the valance, intensity and concordance of verbal behavior (e.g., acoustic features like volume, pitch, prosody, and tone, as well as semantic features including semantic word embedding vectors), the number of real or simulated others who are simultaneously communicating, the presence of shared versus unshared communication goals with and among the real or simulated others, the predictability of these communicative features across time, and the like. Use of the aforementioned semantic word embedding vectors may be based on models such as word2vec and/or alternative embedding algorithms such as GloVe.

As part of step 306, the on-board computing device 204 and/or the support computing device(s) 201 may display results of the evaluation of step 304. The results may be displayed to the subject or another individual, such as clinician. The subject may be rewarded for their improvement (e.g., using a points system). Such rewards may allow the subject to, for example, decorate a space in a scenario.

In step 307, the on-board computing device 204 and/or the support computing device(s) 201 may determine whether to end the presentation of the extended reality environment. Presentation may end at the end of a particular time period, when the subject has completed a particular scenario in the extended reality environment, when the subject has generated a particular value in the therapeutic data or the like. If the answer is yes, the flow chart ends. Otherwise, the flow chart returns to step 302.

### Examples of Different Scenarios

FIG. 4 depicts a diagram representing how different tasks may be presented as part of the extended reality environment presented tin step 302 of FIG. 3. A testing scenario 401, observational scenario 402, learning scenario 403, practical scenario 404, and relaxation scenario 405 are shown. As shown by the arrows in FIG. 4, the different tasks may be interrelated such that, for example, a therapeutic intervention may include moving a subject from the learning scenario 403 to the testing scenario 401, from the testing scenario 401 to the relaxation scenario 405, and the like.

In the testing scenario 401, the on-board computing device 204 and/or the support computing device(s) 201 may determine one or more skills to be trained. For example, a quick series of scenarios may be presented to the subject to test which skills ay be particularly weak and/or otherwise need attention. As another example, the subject, a clinician, and/or a caregiver may be presented with a list of skills to be trained. The testing scenario 401 may be referred to as a “Skill Thermometer” module.

In the observational scenario 402, the on-board computing device 204 and/or the support computing device(s) 201 may provide, via an XR environment presented by the XR device 202, the subject the opportunity to observe symptom-relevant behaviors. For example, the subject may be shown examples of skills that should be developed, examples of interactions where skills were not properly used, or the like. The observational scenario 402 may be referred to as a “Set One” module.

In the learning scenario 403, the on-board computing device 204 and/or the support computing device(s) 201 may provide, via an XR environment presented by the XR device 202, the subject a learning opportunity in the extended reality environment. Such as opportunity may include allowing the subject to test social interactions, practice looking avatars in the eyes, practice speaking, or the like. The learning scenario 403 may be referred to as a “Do One” module.

In the practical scenario 404, the on-board computing device 204 and/or the support computing device(s) 201 may provide, via an XR environment presented by the XR device 202, the subject strategies for transferring skills learned in the extended reality environment to the real world. As part of the practical scenario 404, the subject's ability to transfer such skills to the real world may be evaluated. The practical scenario 404 may be referred to as a “Live One” module.

The practical scenario 404 may include providing the subject with strategies for implementing the skills learned in an extended reality environment. For example, the practical scenario 404 may include adding data corresponding to the skill to a calendar of the subject, such that the subject is reminded to practice the skill at a certain time. As another example, the practical scenario 404 may include transmitting messages (e.g., text messages, e-mails) to a subject to remind them of the skills learned in the extended reality environment. As yet another example, the practical scenario 404 may include checking with a subject before and after a particular task associated with a skill is performed in real life, which may both prepare the subject for the task (e.g., by asking “Are you ready?”) as well as receive feedback, from the subject, regarding the skill (e.g., by asking “How did it go?”).

In the relaxation scenario 405, the on-board computing device 204 and/or the support computing device(s) 201 may provide, via an XR environment presented by the XR device 202, the subject physiological and/or psychological relaxation in a virtual environment. Such a scenario may include providing, to the subject, a room with elements chosen and/or modified by the subject, such that the subject may enjoy the familiarity of the relaxation scenario 405. The relaxation scenario 405 may be referred to as a “My Room” module.

All scenarios depicted in FIG. 4 may be repeated. The intensity of any scenario may be increased based on the results of any other scenario(s). For example, an intensity of the learning scenario 403 may be increased based on determining that results from the practical scenario 404 indicate that the subject is ill-prepared to transfer one or more skills to the real world.

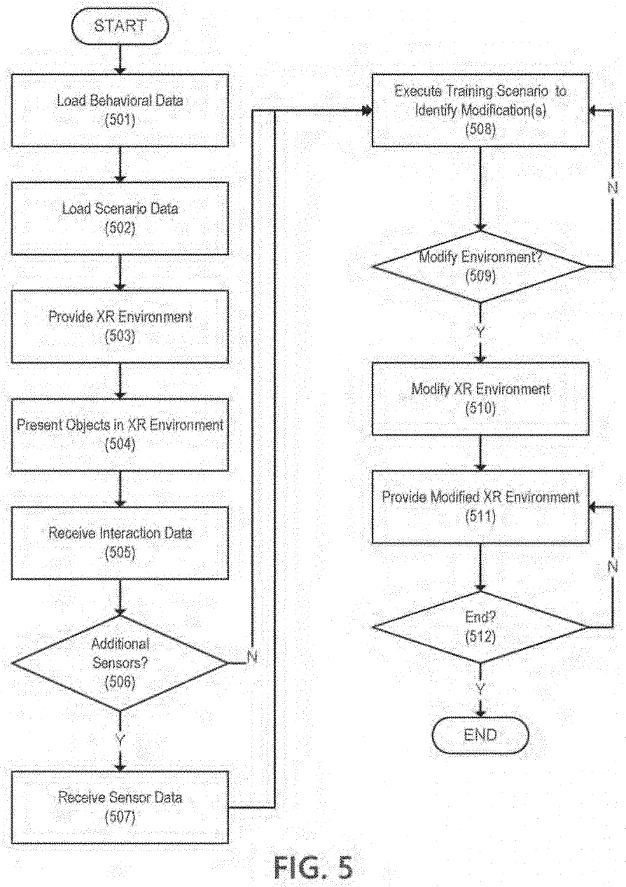

FIG. 5 depicts a flow chart for presenting an extended reality environment. The steps shown in FIG. 3 may be performed to, for example, monitor, assess, and/or treat one or more symptoms of social, communicative, and/or sensory deficits in a tested subject.

In step 501, the on-board computing device 204 and/or the support computing device(s) 201 may load behavioral data. For example, the on-board computing device 204 and/or the support computing device(s) 201 may receive data identifying one or more behaviors associated with symptoms of ASD. The behavioral data may identify and/or otherwise indicate behaviors associated with symptoms of ASD. The data may be received by one or more servers or other sources, such as the database(s) 206. For example, the data may be received from a database, maintained by a clinician, which specifies behaviors associated with symptoms of ASD. The behavioral data may be generated based on analyzing a raw dataset associated with ASD. For example, a dataset of subject data from subjects with ASD may be collected, and such data may be processed to determine commonalities between the subjects.

In step 502, the on-board computing device 204 and/or the support computing device(s) 201 may load scenario data. Such scenario data may be loaded from a database, such as the database(s) 206. Scenario data may be data which defines a scenario (including any tasks to be performed in a scenario), and may comprise indications of a scenario environment (e.g., a location, such as a convenience store), one or more objects in the environment (e.g., avatar objects, chairs, walls, or the like), one or more tasks to be performed in the environment (e.g., finding an item in a convenience store and purchasing it), including whether those tasks should be performed in a particular sequence (e.g., an order of tasks for purchasing an item: locate item, locate cashier, pull out money, pay for item, leave store), and/or data to be measured to assess whether and to what degree the task was completed and any logic for calculating behavioral scores assessing the level/extent of task completion. For example, the on-board computing device 204 and/or the support computing device(s) 201 may receive scenario data specifying a plurality of different tasks, wherein the plurality of different tasks are configured to train skills associated with the one or more behaviors associated with the symptoms of ASD. The scenario data may include a training scenario. The scenario data may indicate parameters for training one or more symptoms of ASD. For example, the scenario data may indicate an appropriate amount of time that individuals without ASD look in the eyes of other humans when speaking with them. The scenario data may include a list of activities e.g., tasks) to complete. For example, the scenario data may indicate that subjects with ASD should practice speaking clearly to improve their confidence in speaking. The scenario data may include the data that indicates one or more thresholds. for example, the scenario data may include data which specifies an acceptable distance (e.g., both a minimum distance and a maximum distance) from which a subject should stand from another human.

The scenario data may include a task sequence. A task sequence may be any ordering of a plurality of tasks for performance by a subject. For example, a task sequence for an item purchasing activity may include first entering a virtual store, then locating an item, then picking up the item, then taking the item to a counter, then speaking to a store clerk, then paying for the item, and then leaving.

The scenario data may include all or portions of the existing data discussed with respect to step 301 of FIG. 3. For example, the scenario data may include information indicating a history of performance by a particular subject, indications of which tasks the subject is skilled at, or the like. Such information may be used, as described further below, to aid in determining subsequent tasks for performance by a subject.

In step 503, the XR deice 202 may provide extended reality environment to a subject. For example, the on-board computing device 204 and/or the support computing device(s) 201 may cause the XR device to present an XR environment to a subject. That XR environment might comprise a scenario (e.g., a scenario defined by the scenario data) with one or more tasks. This step may be the same or similar as step 302 of FIG. 3.

In step 504, the XR device 202 may present objects in a scenario in the extended reality environment. For example, the on-board computing device 204 and/or the support computing device(s) 201 may cause the XR device to present, in the XR environment and to the subject, a scenario comprising at least one first task of the plurality of different tasks specified in the scenario, wherein the at least one first task is configured to train a first skill associated with improvement of the one or more behaviors associated with the symptoms of ASD, and wherein the at least one first task is configured to prompt the subject to interact with one or more objects in the XR environment. For example, the scenario data may correspond to training a subject in social interactions, and the scenario presented in the XR environment based on the scenario data may comprise one or more tasks associated with purchasing an item from a store clerk. Presenting an object in an extended reality environment may include displaying one or more visual representations of the object. For example, in the aforementioned scenario, a variety of visual representations of objects might be described, a store clerk, store shelves, a counter, virtual money, and the like. For example, presenting an object in an extended reality environment may include the on-board computing device 204 and/or the support computing device(s) 201 determining a three-dimensional model corresponding to the object and displaying it in the extended reality environment. Returning to the above example, such three-dimensional models might correspond to the objects above, such that the XR environment is configured to appear as a real three dimensional store. Presenting the objects need not mean that the objects are always presented to the subject. For example, if the subject looks away from the object in the extended reality environment, the object may be removed from the environment for processing efficiency purposes. As such, for example, and returning to the above scenario example, turning away from the store clerk may cause the XR environment to cease rendering the store clerk so as to preserve processing resources.

Presenting the objects may be part of presenting, by the XR device 202 and in a scenario in the extended reality environment, a task, such as the tasks discussed with respect to FIGS. 3 and 4. The task may be one or a plurality of different tasks specified by, e.g., the scenario data. The task may be configured to train a skill associated with improvement of the one or more behaviors associated with symptoms of ASD. The task may prompt the subject to interact with the one or more objects. The skill may correspond to one or more of: speech patterns of the subject, eye gaze of the subject, a location of the subject, and/or movement of the subject.

The XR device 202 may cause the extended reality environment to present, in a scenario, multiple tasks at once. For example, presenting the objects may be part of presenting multiple tasks, such as a first task (e.g., one for training symptoms of ASD) and a daily living task (e.g., one that the subject must complete during the first task). In this manner, steps discussed herein might involve multiple tasks, which the subject may be tasked with performing in sequence or in parallel. As will be detailed further below, this may advantageously allow for testing of one task (e.g., one associated with training symptoms of ASD) while tasking a subject with performing a series of other simulated tasks (e.g., everyday living tasks).

As an example of the objects, the XR device 202 may present, via the extended reality environment (and, e.g., in a scenario), an avatar object. The subject may be prompted to interact with the avatar object by, for example, talking to the avatar object, positioning themselves around the avatar object, or the like. Performance of such interactions (e.g., successful conversations, shaking the hand of the avatar successfully) might be monitored by the XR device 202.

In step 505, the on-board computing device 204 and/or the support computing device(s) 201 may receive interaction data. For example, the on-board computing device 204 and/or the support computing device(s) 201 may detect a subject interaction with the one or more objects in a scenario presented in the XR environment. Interaction data may include any data which indicates interaction with the subject with any portion of the extended reality environment. Interaction data may additionally and/or alternatively be referred to as subject data. Interaction data may include, for example, where the subject stands or moves, where the subject looks (e.g., eye gaze), speech or other sounds made by the subject, or the like.

The interaction data may include data that is the same or similar as the therapeutic data described with respect to step 303 of FIG. 3. For example, the interaction data may include a variety of information about a subject, such as their speech patterns, where their eyes are looking, their heart rate, or the like.

The interaction data may be based on detecting a subject interacting with one or more objects in the extended reality environment (e.g., in a scenario). The subject may, for example, move an object, speak to an object, look at an object, or otherwise perform any action or inaction with respect to an object.

The interaction data need not include information about a subject affirmatively interacting with any object. For example, the subject not interacting with an object may, itself, be valuable data. For example, a subject staring at the floor and not interacting with an avatar object may itself be a valuable data point for the purposes of determining whether the subject is accomplishing an assigned task within the scenario (e.g., creating and holding eye contact with a conversation partner).

As discussed briefly with regard to step 303 of FIG. 3, the on-board computing device 204 and/or the support computing device(s) 201 receiving the interaction data may additionally an/or alternatively include collecting eye tracking data. Such eye tracking data may be collected using one or more eye tracking systems of an extended reality device. For example, the eye tracking data may be collected by the camera(s) 203d and/or the position tracking elements 203e. Using the eye tracking data, gaze data may be generated by identifying one or more second objects in a scenario which the subject looked at during performance of the task. For example, the eye tracking data may be compared to positions of objects in a scenario presented by an extended reality environment to determine whether the subject was looking at the objects. The gaze data may thereby indicate whether a subject looked at a particular region (e.g., the eyes, the feet) of one or more second objects (e.g., an avatar object). In this manner, using the gaze data, it may be determined whether the subject looked at a socially appropriate portion of an object (e.g., the eyes of an avatar object) or a socially inappropriate portion of an object (e.g., the chest of an avatar object). Similarly, the gaze data may indicate whether a subject looked away from one or more objects for a period of time. In this way, the gaze data might indicate that the subject avoided looking at a conversation partner. As such, any of the information may be inputs for a scenario (e.g., the scenario received in step 502) which may be used to determine whether and/or how to modify a scenario (e.g., an XR environment presented by the XR device 202). For instance, the on-board computing device 204 and/or the support computing device(s) 201 may use the scenario to determine whether the eye tracking data indicates symptoms of ASD.

As also discussed briefly with regard to step 303 of FIG. 3, the on-board computing device 204 and/or the support computing device(s) 201 receiving the interaction data may additionally and/or alternatively include collecting voice data. The voice data may correspond to vocal interaction, by a subject with one or more objects in a scenario presented in an extended reality environment. For example, the voice data may correspond to speech, by the subject, directed to an avatar object. The voice data may be received by microphones, such as may be part of the audio device(s) 203b. Based on the voice data, a confidence score may be calculated. The confidence score may be associated with a confidence of the subject when speaking. For example, excessive pauses or words evocate of uncertainty might be associated with a relatively low confidence score, whereas clear, concise, and sufficiently loud speech might be associated with a relatively high confidence score. Based on the voice data, a clarity score may be calculated. The clarity score may be associated with a clarity of the subject when speaking. For example, quiet speech might be provided a relatively low clarity score, whereas sufficiently loud and defined speech might be provided a relatively high clarity score. As such, any of this information may be inputs for a scenario (e.g., the scenario received in step 502) which may be used to determine whether and/or how to modify a scenario (e.g., an XR environment presented by the XR device 202). For instance, this on-board computing device 204 and/or the support computing device(s) 201 may use the scenario to determine whether the voice data indicates symptoms of ASD.

In step 506, the on-board computing device 204 and/or the support computing device(s) 201 may determine whether there are additional sensors. The additional sensors may include any sensor device which might provide detail in addition to the interaction data received in step 505. For example, the additional sensors may include a biometric sensor, such as the biometric tracking device(s) 205. If the are additional sensors, the flow chart proceeds to step 507. Otherwise, the flow chart proceeds to step 508.

In step 507, if there are additional sensors, the on-board computing device 204 and/or the support computing device(s) 201 may receive sensor data. For example, the on-board computing device 204 and/or the support computing device(s) 201 may receive, from one or more biometric tracking devices, biometric data that is associated with the subject and collected during performance of the at least one first task, wherein calculating the behavioral score includes calculating the behavioral score further based on the biometric data. The sensor data may be associated with the subject. For example, the sensor data may include an indication of the heart rate of the subject. The sensor data collected may be the same or similar as all or portions of the therapeutic data discussed with respect to step 303 of FIG. 3. As such, any of this sensor data may be inputs for a scenario (e.g., the scenario received in step 502) which may be used to determine whether and/or how to modify a scenario (e.g., an XR environment presented by the XR device 202). For instance, the on-board computing device 204 and/or the support computing device(s) 201 may use the scenario to determine whether the sensor data indicates symptoms of ASD.

The sensor data may include biometric data, received from one or more biometric tracking devices, corresponding to a subject and collected during performance of a task. Biometric data may include, for example, a heart rate of a subject, oxygen levels of the subject, or the like. Such information may indicate, for example, a nervousness of the subject or engagement of the subject.

In step 508, the on-board computing device 204 and/or the support computing device(s) 201 may execute the scenario data to identify modifications to the extended reality environment (e.g., a scenario presented in the extended reality environment). This step may be the same or similar as step 305 of FIG. 3. The modifications may be based on the interaction data, the sensor data, and/or the scenario data. The scenario data may include a task sequence to be performed by a subject. For example, a task sequence may specify that, to train interactions of a subject in a store, the subject may be prompted to first enter the store, then locate an item, then bring an item to the cashier, then pay for the item. In other words, based on a task sequence, a next step of the task sequence may be identified, and a modification may be determined based on the next step of the task sequence.

As part of identifying modifications to the extended reality environment, the on-board computing device 204 and/or the support computing device(s) 201 may calculate a behavioral score indicative of (e.g., which is generated based on data indicating) one or more behaviors associated with the symptoms of ASD). For example, the on-board computing device 204 and/or the support computing device(s) 201 may calculate, based on the one or more subject interactions, a behavioral score indicated of (e.g., that provides an objective measure of) the one or more behaviors associated with the symptoms of ASD. Calculating the score may include calculating performance metrics corresponding to the subject interaction. For instance, the performance metric may correspond to how quickly a subject responded to a question, how close or far they stood from an avatar object, or the like. As another example, a computing device may calculate a behavioral score that comprises a ranking of how often a particular subject exhibits behaviors potentially associated with symptoms of ASD, and the performance metric may be associated with those behaviors. If biometric data was received, then calculating the behavioral score may include the on-board computing device 204 and/or the support computing device(s) 201 comparing the biometric data to a standard established by the scenario data. For example, the scenario data may include an indication that a heart rate over 100 beats per minute indicates nervousness, and heart rate from the biometric data may be compared to this 100 beats per minute threshold.

The on-board computing device 204 and/or the support computing device(s) 201 identifying modifications to the extended reality environment (e.g., the scenario presented in the extended reality environment) may include selecting a second task. The task may be selected from a plurality of different tasks specified by the scenario data. Selecting the task may be based on the behavioral score. The second task may be the same or different from the task(s) originally presented to a subject. For example, the subject might first be presented with two tasks (e.g., talking to a cashier and opening a wallet to locate a credit card), and the modification may include presenting a second task that replaces one of the two tasks (e.g., such that the subject is tasked with still talking to the cashier while paying for a good using a point-of-sale system). As such, selecting the second task may comprise replacing one task of a scenario, while also keeping another task of the same scenario going.

The second task may be selected based on performance of the subject of one or more previous tasks. A subject might perform one or more tasks well or poorly, as indicated, for example, by the interaction data discussed above. Along those lines, the mere fact that a subject completed a task need not indicate that the subject performed the task well, and need not indicate that the subject is addressing symptoms associated with ASD. As such, a second task may be selected based on a quality of performance of one or more previous tasks. For example, if a subject is doing particularly well with past tasks, the second task may be selected to increase a scenario difficulty level. Such a scenario difficulty level may be a sum of difficulty levels of a plurality of tasks presented in a scenario. For example, for a scenario involving walking into a party, the task of opening the door might be relatively easy, but the next task (e.g., saying hello to someone at the party) might be significantly harder for an individual experiencing ASD, such that the scenario difficulty level may be relatively high. Conversely, if a subject is doing poorly with past tasks the second task may be selected to decrease a scenario difficulty level. There also may be instances in which one or more previous tasks were completed, but the quality of the subject's performance of those previous tasks was average and/or as predicted. In such a circumstance, the second task might be selected based on a logical sequence of tasks, which may be defined by the scenario data. For example, if the scenario data indicates a logical sequence of three tasks and the subject performs the first task well (but not particularly well or poorly), the next task in the sequence may be selected accordingly.

The second task may additionally and/or alternatively be selected based on interaction data, such as behavioral scores. The interaction data of a subject (e.g., with respect to one or more symptoms of ASD) may be used to select a second task such that the second task trains, ameliorates, and/or otherwise addresses symptoms of ASD. For example, if a subject completed a task well and had positive behavioral scores (e.g., which indicated that the subject did not behave in a manner that reflected symptoms of ASD), the second task may be selected to be slightly harder, so as to train the subject. In contrast, if the subject completed a task well but had negative behavioral scores (e.g., which indicated that the subject behaved in a manner that reflected difficulty with handling symptoms of ASD), then the second task might be selected to be slightly easier, so as to allow the subject to take a break.

The second task may be selected based on feedback from the subject. A subject might provide feedback regarding a scenario and/or one or more tasks of the scenario. For example, the feedback might indicate that the subject is feeling confident, worried, overwhelmed, or the like. Such feedback might be received via the on-board computing device 204 and/or the support computing device(s) 201 by, for example, presenting prompts in the XR environment, receiving verbal feedback from the subject, or the like. Based on the feedback, the second task may be selected. For example, if the subject is feeling confident, a relatively more difficult second task may be selected. As another example, if the subject is feeling worried and/or overwhelmed, a relatively easier task may be selected.

In step 509, the on-board computing device 204 and/or the support computing device(s) 201 may determine whether to modify the extended reality environment (e.g., by modifying a scenario presented by the extended reality environment). For example, the on-board computing device 204 and/or the support computing device(s) 201 may select from the plurality of different tasks and based on the behavioral score indicative of the one or more behaviors associated with the symptoms of ASD, at least one second task of the plurality of different tasks, wherein the at least one second task is configured to train a second skill associated with improvement of the one or more behaviors associated with the symptoms of ASD, the second skill being different from the first skill. Determining whether to modify the extended reality environment may be based on the scenario data. For example, if the scenario data specifies a task sequence, then determining whether to modify the extended reality environment may include determining that one task is completed, such that a next task (e.g., a next task in the task sequence) should be presented to a subject. If it is determined to modify the extended reality environment, the flow chart proceeds to step 510. Otherwise, the flow chart returns to step 508.

In step 510, the on-board computing device 204 and/or the support computing device(s) 201 may cause the extended reality environment presented by the XR device 202 to be modified. For example, the on-board computing device 204 and/or the support computing device(s) 201 may modify, base on the scenario, the XR environment to present the second task in a scenario. The extended reality environment may be modified based on the modifications identified in step 508. This step may be the same or similar as step 306 of FIG. 3. For example, if the modification includes presenting a second task to the subject, then the modification may include modifying the extended reality environment to present the second task in the same or a different scenario. As another example, if the modification comprises making a current task easier, then the extended reality environment may be modified to lower a difficulty of one or more portions of the current task.

In step 511, the XR device may provide the modified extended reality environment to the subject. This step may be the same or similar as step 503 and/or step 302 of FIG. 3. As part of presenting the modified extended reality environment, an updated behavioral score may be determined. Such an updated behavioral score may involve the same or similar steps as described with respect to step 508 of FIG. 5.

Steps 508 through 511 may be performed without involvement of a human. In other words, the steps may be performed automatically, and based on logic provided by the scenario data. In this manner, a clinician need not be involved in the process of modifying a training program for a subject. This may advantageously allow a subject to proceed through multiple tasks (e.g., multiple tasks in parallel, a task sequence) without affirmative monitoring and intervention by another human being.

In step 512, the on-board computing device 204 and/or the support computing device(s) 201 may determine whether to end presentation of the extended reality environment. The on-board computing device 204 and/or the support computing device(s) 201 may determine to end presentation of the extended reality environment if, for example, a subject has completed one or more tasks of one or more scenarios. For example, if the modified extended reality environment prompts a subject to complete two tasks in parallel, the on-board computing device 204 and/or the support computing device(s) 201 may determine to end presentation of the extended reality environment based on a determination that both of the tasks have been completed. Additionally and/or alternatively, the on-board computing device 204 and/or the support computing device(s) 201 may determine to end presentation of the extended reality environment based on the interaction data and/or the sensor data. For example, if the subject appears to be fired, distracted, or otherwise exhausted, the on-board computing device 204 and/or the support computing device(s) 201 may determine to end presentation of the extended reality environment. If it is determined to end the extended reality environment, the flow chart ends. Otherwise, the flow chart returns to step 511.

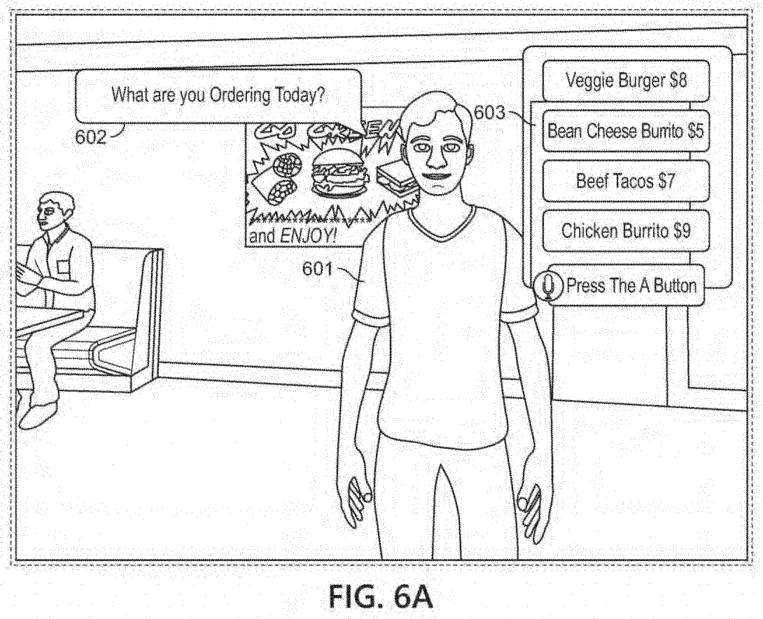

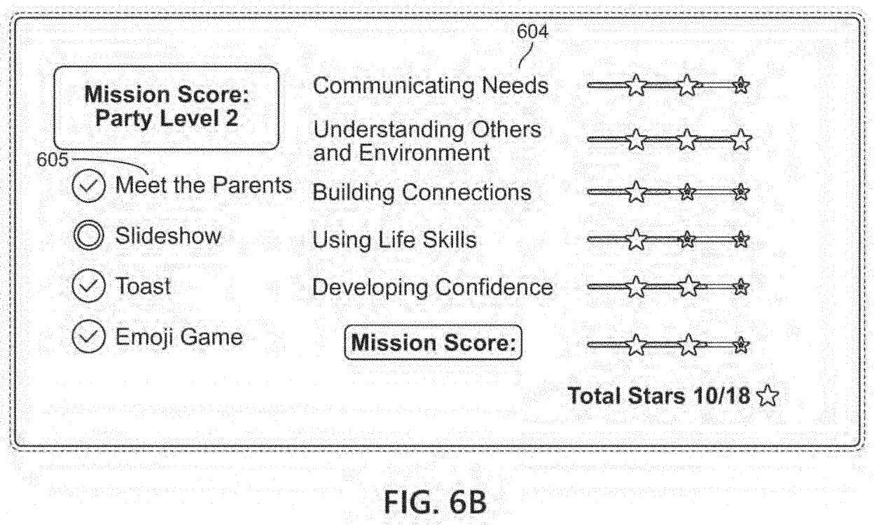

FIG. 6A shows an example of an extended reality environment which may be generated, presented and/or otherwise provided by the XR device 202. In particular, FIG. 6A shows an example extended reality environment where a subject is prompted to order food. The extended reality environment in FIG. 6A comprises a human-like avatar object 601 which asks, in a text bubble 602, what the subject would like to order. The subject is presented with various options 603, which the subject may speak by pressing the “A” button. The XR environment also comprises a variety of distractions, such as a poster behind the avatar object and another avatar objected seated at a booth. In the example depicted by FIG. 6A, a subject might be tasked with speaking one of the options 603 rather than, for example, looking away, walking away from the avatar object, or the like.

The extended reality environment depicted in FIG. 6A may be all or portions of the environment presented in step 302 of FIG. 3 and/or the environment provided in step 503 of FIG. 5. For example, the extended reality environment depicted in FIG. 6A may be part of an extended reality environment of a food purchase scenario. In such an example, step 503 of FIG. 5, which relates to presenting objects in an XR environment, might comprise rendering the wall shown in FIG. 6A, the booth shown in FIG. 6A, the avatar object 601 shown in FIG. 6A, or the like. In turn, in that example, and as part of step 505 (which involves receiving interaction data), the system may receive interaction data which indicates interactions with any one of these objects, for example, the subject may stand near a wall, speak with the avatar object 601, or the like.

FIG. 6B shows an example of output associated with behaviors, by a subject, in an XR environment. In particular, the output (which may, e.g., be presented and/or otherwise provided by a computing device, such as XR device 202) shown in FIG. 6B depicts a series of scores 604 relating to various strategies for ameliorating symptoms of ASD. The scores 604 are scored from zero to three stars, with the total number of stars displayed at the bottom of the output. On the left-hand side of the output, a list 605 of tasks completed by a subject are displayed. For example, as shown in the example in FIG. 6B, the subject completed tasks involving meeting parents, toasting, and playing an emoji game, but failed to participate in a slideshow task.

The output shown in FIG. 6B might be part of step 508 of FIG. 5 in that it may relate to the execution of a training scenario and the identification of modifications to make to the extended reality environment. For example, the output shown in FIG. 6B indicates that the subject performed somewhat poorly with respect to “Building Communications” and “Using Life Skills.” As such, a second task selected for performance by the subject might comprise, for example, a task associated with training skills for “Building Communications” and/or “Using Life Skills.”

The following paragraphs (M1) through (M20) describe examples of methods that may be implemented in accordance with the present disclosure.

(M1) A method for assessing Autism Spectrum Disorder (ASD), the method comprising: at a computing device that comprises at lease one processor and memory and that is coupled to an extended reality (XR) device; receiving data identifying one or more behaviors associated with symptoms of ASD for a subject; receiving, based on the one or more behaviors, a scenario specifying a plurality of different tasks, wherein the plurality of different tasks are configured to train skills associated with the symptoms of ASD; causing the XR device to present, based on the scenario, an XR environment, causing the XR device to present, in the XR environment and to the subject, a first task of the different tasks specified in the scenario, wherein the first task is configured to train a first skill associated with improvement of at least one of the behaviors associated with the symptoms of ASD, and wherein the first task is configured to prompt the subject to interact with an object in the XR environment; detecting a subject interaction with the object in the XR environment; generating, based on the subject interaction, interaction data that indicates performance, by the subject, of the one or more behaviors associated with the symptoms of ASD; selecting, from the different tasks and based on the interaction data, a second one of the different tasks, wherein the second task is configured to train a second skill associated with improvement of another one or more of the behaviors associated with the symptoms of ASD, the second skill being different from the first skill; and modifying, based on the scenario the XR environment to present the second task.

(M2) The method of claim 1, further comprising: receiving, from one or more biometric tracking devices, biometric data that is associated with the subject and collected during performance of the first task, wherein generating the interaction data comprises generating the interaction data further based on the biometric data.

(M3) The method of claim 2, wherein calculating the score associated with the one or more behaviors comprises comparing the biometric data to a standard established by the scenario.

(M4) The method of any one of claims 1-3, wherein calculating the score associated with the one or more behaviors comprises calculating performance metrics, corresponding to the subject interaction, that indicate how well the subject performs the one or more behaviors in the XR environment.

(M5) The method of any one of claims 1-4, wherein selecting the second task is responsive to use input indicating that the user is unresponsive to the first task, and wherein modifying the XR environment comprises presenting the second task without human interaction.

(M6) The method of any one of claims 1-5, wherein the scenario specifies a task sequence comprising a plurality of different tasks to be performed in an order, and wherein selecting the second task is based on determining a task, of the plurality of different tasks and based on the order, to be performed after the first task.

(M7) The method of any one of claims 1-6, further comprising: generating, after modifying the XR environment to present the second task, updated interaction data based on further subject interactions in response to the second task.

(M8) The method of any one of claims 1-7, wherein the first skill corresponds to one or more of: speech patterns of the subject; eye gaze of the subject; a location of the subject as compared to a location of an avatar object; a decision made in the XR environment; or movement of the subject.

(M9) A method for assessing Autism Spectrum Disorder (ASD), the method comprising, at a computing device that comprises at least one processor and memory and that is coupled to an extended reality (XR) device; receiving data identifying one or more behaviors associated with symptoms of ASD for a subject; receiving, base d on the one or more behaviors, a scenario specifying a plurality of different tasks, wherein the plurality of different tasks are configured to train skills associated with the symptoms of ASD, causing the XR device to present, based on the scenario, an XR environment; causing the XR device to present, in the XR environment and to the subject, a first task of the different tasks specified in the scenario, wherein the first task is configured to train a first skill associated with improvement of at least one of the behaviors associated with the symptoms of ASD, and wherein the first task is configured to prompt the subject to interact with an object in the XR environment; detecting one or more interactions with the one or more objects in the XR environment, the one or ore interactions being associated with the subject; collecting eye tracking data by monitoring, using an eye tracking system of the XR device, eye motions of the subject, generating gaze data by identifying, based on the eye tracking data, one or more second objects in the XR environment which the subject looked at during performance of the task; generating, based on the one or more interactions and based on the gaze data, interaction data that indicates performance, by the subject of the one or more behaviors associated with the symptoms of ASD; selecting, from the different tasks and based on the interaction data, a second one of the different tasks, wherein the second task is configured to train a second skill associated with improvement of another one or more of the behaviors associated with the symptoms of ASD, the second skill being different from the first skill; and modifying, based on the scenario, the XR environment to present the second task.

(M10) The method of claim 9, further comprising: generating, after modifying the XR environment to present the second task, an updated interaction data based on further subject interactions in response to the second task.

(M11) The method of claim 9 or claim 10, wherein the gaze data indicates whether the subject looked at a particular region of the one or more second objects.

(M12) The method of claim 11, wherein the one or more second objects comprise an avatar object, and wherein the particular region comprises eyes of the avatar object.

(M13) The method of any one of claims 9-12, wherein the gaze data indicates whether the subject looked away from the one or more second objects for a period of time.

(M14) A method for assessing Autism Spectrum Disorder (ASD). the method comprising: at a computing device that comprises at least one processor and memory and that is coupled to an extended reality (XR) device; receiving data identifying one or more behaviors associated with symptoms of ASD for a subject; receiving, based on the one or more behaviors, a scenario specifying a plurality of different tasks, wherein the plurality of different tasks are configured to train skills associated with the symptoms of ASD; causing the XR device to present, based on the scenario, an XR environment; causing the XR device to present, in the XR environment and to the subject, a first task of the different tasks specified in the scenario, wherein the first task is configured to train a first skill associated with improvement of at least one of the behaviors associated with the symptoms of ASD, and wherein the first task is configured to prompt the subject to interact with an object in the XR environment; detecting one or more interactions with the one or more objects in the XR environment, the one or more interactions being associated with the subject; receiving, via one or more microphones f the XR device, voice data corresponding to vocal interaction, by the subject, with the one or more objects in the XR environment; generating, based on the subject interaction, interaction data that indicates performance, by the subject, of the one or more behaviors associated with the symptoms of ASD; selecting, from the different tasks and based on the interaction data, a second one of the different tasks; wherein the second task is configured to train a second skill associated with improvement of another one or more of the behaviors associated with the symptoms of ASD, the second skill being different from the first skill; and modifying, based on the scenario, the XR environment to present the second task.

(M15) The method of claim 14, further comprising: generating, after modifying the XR environment to present the second task, updated interactions data based on further subject interactions in response to the second task.

(M16) The method of claim 14 or claim 15, wherein generating the interaction data comprises: calculating, based on the voice data, a confidence score associated with a confidence of the subject when speaking.

(M17) The method of any one of claims 14-16, wherein generating the interaction data comprises: calculating based on the voice data, a clarity score associated with a clarity of speech of the subject.

(M18) A method for assessing Autism Spectrum Disorder (ASD), the method comprising: at a computing device that comprises at least one processor and memory and that is coupled to an extended reality (XR) device; receiving data identifying one or more behaviors associated with symptoms of ASD for a subject; receiving, based on the one or more behaviors, a scenario specifying a plurality of different tasks, wherein the plurality of different tasks are configured to train skills associated with the symptoms of ASD; causing the XR device to present, based on the scenario, an XR environment; causing the XR device to present, in the XR environment and to the subject, a first task of the plurality of different tasks, wherein the first task is configured to train a first skill associated with improvement of at least one of the behaviors associated with the symptoms of ASD; causing the XR device to present, in the XR environment and to the subject, a second task of the plurality of different tasks, wherein the second task is configured to emulate a daily living skill; collecting subject data by monitoring performance, by the subject, of the first task and the second task over a time period; generating based on the subject interaction, interaction data that indicates performance, by the subject, of the one or more behaviors associated with the symptoms of ASD; selecting, from a plurality of different tasks and based on the interaction data, a third task configured to train a second skill associated with improvement of at least one of the behaviors associated with the symptoms of ASD; and causing the XR device to modify, based on the template, the XR environment to present the second task and the third task.

(M19) The method of claim 18, wherein causing the XR device to modify the XR environment comprises: causing the XR environment to present the second task for performance, by the subject, in parallel with the third task.

(M20) The method of claim 18 or claim 19, wherein causing the XR device to present the second task comprises causing the XR device to present the second task in parallel with the first task.

The following paragraphs (A1) through (A23) describe examples of apparatuses that may be implemented in accordance with the present disclosure.

(A1) An apparatus, coupled to an extended reality (XR) device, for assessing Autism Spectrum Disorder (ASD), the apparatus comprising: one or more processors, and memory storing instructions that, when executed by the one or more processors, cause the apparatus to: receive data identifying one or more behaviors associated with symptoms of ASD for a subject; receive, based on the one or more behaviors, a scenario specifying a plurality of different tasks, wherein the plurality of different tasks are configured to train skills associated with the symptoms of ASD; cause the XR device to present, based on the scenario, an XR environment; cause the XR device to present, in the XR environment and to the subject, a first task of the different tasks specified in the scenario, wherein the first task is configured to train a first skill associated with improvement of at least one of the behaviors with the symptoms of ASD, and wherein the first task is configured to prompt the subject to interact with an object in the XR environment; detect a subject interaction with the object in the XR environment; generate based on the subject interaction, interaction data that indicates performance, by the subject, of the one or more behaviors associated with the symptoms of ASD; select, from the different tasks and based on the interaction data, a second one of the different tasks, wherein the second task is configured to train a second skill associated with improvement of another one or more of the behaviors associated with the symptoms of ASD; the second skill being different from the first skill; and modify, based on the scenario, the XR environment to present the second task.

(A2) The apparatus of (A1), wherein the instructions, when executed by the one or more processors, cause the apparatus to: receiving from one or more biometric tracking devices, biometric data that is associated with the subject and collected during performance of the first task, wherein the instructions, when executed by the one or more processors, cause the apparatus to generate the interaction data further based on the biometric data.

(A3) the apparatus of (A2), wherein the instructions, when executed by the one or more processors, cause the apparatus to calculate the score associated with the one or more behaviors based on comparing the biometric data to a standard established by the scenario.

(A4) The apparatus of any one of (A1)-(A3), wherein the instructions, when executed by the one or more processors, cause the apparatus to calculate the score associated with the one or more behaviors based on calculating performance metrics, corresponding to the subject interaction, that indicate how well the subject performs the one or more behaviors in the XR environment.

(A5) The apparatus of any one of (A1)-(A4), wherein the instructions, when executed by the one or more processors, cause the apparatus to select the second task responsive to use input indicating that the user is unresponsive to the first task, and wherein the instructions, when executed by the one or more processors, cause the apparatus to modify the XR environment by presenting the second task without human interaction.

(A6) The apparatus of any one of (A1)-(A5), wherein the scenario specifies a task sequence comprising a plurality of different tasks to be performed in an order; and wherein the instructions, when executed by the one or more processors, cause the apparatus to select the second task is based on determining a task, of the plurality of different tasks and based on the order, to be performed after the first task.

(A7) The apparatus of any one of (A1)-(A6), wherein the instructions, when executed by the one or more processors, cause the apparatus to: generate, after modifying the XR environment to present the second task, updated interaction data based on further subject interactions in response to the second task.

(A8) The apparatus of any one of (A1)-(A7), wherein the first sill corresponds to one or more of: speech patterns of the subject: eye gaze of the subject; a location of the subject as compared to a location of an avatar object; a decision made in the XR environment; or movement of the subject.

(A9) An apparatus, coupled to an extended reality (XR) device, for assessing Autism Spectrum Disorder (ASD), the apparatus comprising: one or more processors, and memory storing instructions that, when executed by the one or more processors, cause the apparatus to: receive data identifying one or more behaviors associated with symptoms of ASD for a subject, receive, based on the one or more behaviors, a scenario specifying a plurality of different tasks, wherein the plurality of different tasks are configured to train skills associated with the symptoms of ASD; cause the XR device to present, based on the scenario, an XR environment, cause the XR device to present, in the XR environment and to the subject, a first task of the different tasks specified in the scenario, wherein the first task is configured to train a first skill associated with improvement of at least one of the behaviors associated with the symptoms of ASD, and wherein the first task is configured to prompt the subject to interact with an object in the XR environment; detect one or more interactions with the one or more objects in the XR environment, the one or more interactions being associated with the subject; collect eye tracking data by monitoring, using an eye tracking system of the XR device, eye motions of the subject; generate gaze data by identifying, based on the eye tracking data, one or more second objects in the XR environment which the subject looked at during performance of the task; generate, based on the one or more interactions and based on the gaze data, interaction data that indicates performance, by the subject, of the one or more behaviors associated with the symptoms of ASD; select, from the different tasks and based on the interaction data, a second one of the different tasks, wherein the second task is configured to train a second skill associated with improvement of another one or more of the behaviors associated with the symptoms of ASD, the second skill being different from the first skill; and modify, based on the scenario, the XR environment to present the second task.

(A10) the apparatus of (A10), wherein the instructions, when executed by the one or more processors, cause the apparatus to: generate, after modifying the XR environment to present the second task; an updated interaction data based on further subject interactions in response to the second task.

(A11) The apparatus of (A9) or (A10), wherein the gaze data indicates whether the subject looked at a particular region of the one or more second objects.

(A12) The apparatus of (A11), wherein the one or more second objects comprise an avatar object, and wherein the particular region comprises eyes of the avatar object.

(A13) The apparatus of any one of (A9)-(12), wherein the gaze data indicates whether the subject looked away from the one or more second objects for a period of time.

(A14) An apparatus, coupled to an extended reality (XR) device, for assessing Autism Spectrum Disorder (ASD), the apparatus comprising: one or more processors; and memory storing instructions that, when executed by the one or more processors, cause the apparatus to: receive data identifying one or more behaviors associated with symptoms of ASD for a subject; receive, base on the one or more behaviors, a scenario specifying a plurality of different tasks, wherein the plurality of different tasks are configured to train skills associated with the symptoms of ASD; cause the XR device to present, based on the scenario, an XR environment; cause the XR device to present, in the XR environment and to the subject, a first task of the different tasks specified in the scenario, wherein the first task is configured to train a first skill associated with the improvement of at least one of the behaviors associated with the symptoms of ASD, and wherein the first task is configured to prompt the subject to interact with an object in the XR environment; detect one or more interactions with the one or more objects in the XR environment, the one or more interactions being associated with the subject; receive, via one or more microphones of the XR device, voice data corresponding to vocal interaction, by the subject, with the one or more objects in the XR environment; generate, based on the subject interaction, interaction data that indicates performance, by the subject, of the one or more behaviors associated with the symptoms of ASD; select, from the different tasks and based on the interaction data, a second one of the different tasks, wherein the second task is configured to train a second skill associated with improvement of another one or more of the behaviors associated with the symptoms of ASD, the second skill being different from the first skill, and modify, based on the scenario, the XR environment to present the second task.

(A15) The apparatus of (A14), wherein the instructions, when executed by the one or more processors, cause the apparatus to: generate, after modifying the XR environment to present the second task, updated interaction data based on further subject interactions in response to the second task.

(A16) The apparatus of (A14) or (A15), wherein the instructions, when executed by the one or more processors, cause the apparatus to generate the interaction data by causing the apparatus to calculate, based on the voice data, a confidence score associated with a confidence of the subject when speaking.

(A17) The apparatus of any one of (A14)-(A16), wherein the instructions, when executed by the one or more processors, cause the apparatus to generate the interaction data by causing the apparatus to: calculate, based on the voice data, a clarity score associated with a clarity of speech of the subject.

(A18) An apparatus, coupled to an extended reality (XR) device, for assessing Autism Spectrum Disorder (ASD), the apparatus comprising: one or more processors; and memory storing instructions that, when executed by the one or more processors, cause the apparatus to: receive data identifying one or more behaviors associated with symptoms of ASD for a subject; receive, based on the one or more behaviors, a scenario specifying a plurality of different tasks, wherein the plurality of different tasks are configured to train skills associated with the symptoms of ASD; cause the XR device to present based on the scenario, an XR environment; cause the XR device to present, in the XR environment and to the subject, a first task of the plurality of different tasks, wherein the first task is configured to train a first skill associated with improvement of at least one of the behaviors associated with the symptoms of ASD; cause the XR device to present, in the XR environment and to the subject, a second task of the plurality of different tasks, wherein the second task is configured to emulate a daily living skill, collect subject data by monitoring performance, by the subject, of the first task, and the second task over a time period; generate, based on the subject interaction, interaction data that indicates performance, by the subject, of the one or more behaviors associated with the symptoms of ASD; select, from a plurality of different tasks and based on the interaction data, a third task configured to train a second skill associated with improvement of at least one of the behaviors associated with the symptoms of ASD, and cause the XR device to modify, based on the template, the XR environment to present the second task and the third task.

(A19) The apparatus of (A18), wherein the instructions, when executed by the one or more processors, cause the apparatus to cause the XR device to modify the XR environment by causing the XR environment to present the second task for performance, by the subject, in parallel with the third task.

(A20) The apparatus of (A18) or (A19), wherein the instructions, when executed by the one or more processors, cause the apparatus to cause the XR device to present the second task by causing the XR device to present the second task in parallel with the first task.

The following paragraphs (CRM1) through (CRM20) describe examples of computer-readable media that may be implemented in accordance with the present disclosure.

(CRM1) One or more non-transitory computer-readable media comprising instructions that, when executed by at least one processor of an computer-readable media, coupled to an extended reality (XR) device, for assessing Autism Spectrum Disorder (CRMSD), cause the computer-readable media to: receive data identifying one or more behaviors associated with symptoms of ASD for a subject; receive, based on the one or more behaviors, a scenario specifying a plurality of different tasks, wherein the plurality of different tasks are configured to train skills associated with the symptoms of ASD; cause the XR device to present, based on the scenario, an XR environment; cause the XR device to present, in the XR environment and to the subject, a first task of the different tasks specified in the scenario, wherein the first task is configured to train a first skill associated with improvement of at least one of the behaviors associated with the symptoms of ASD, and wherein the first task is configured to prompt the subject to interact with an object in the XR environment; detect a subject interaction with the object in the XR environment; generate, based on the subject interaction, interaction data that indicates performance, by the subject, of the one or more behaviors associated with the symptoms of ASD; select, from the different tasks and based on the interaction data, a second one of the different tasks, wherein the second task is configured to train a second skill associated with improvement of another one or more of the behaviors associated with the symptoms of ASD, the second skill being different from the first skill; and modify, based on the scenario, the XR environment to present the second task.

(CRM2) The computer-readable media of (CRM1), wherein the instructions, when executed by the one or more processors, cause the computer-readable media to receive, from one or more biometric tracking devices, biometric data that is associated with the subject and collected during performance of the first task, wherein the instructions, when executed by the one or more processors, cause the computer-readable media to generate the interaction data further based on the biometric data.

(CRM3) The computer-readable media of (CRM2), wherein the instructions, when executed by the one or more processors, cause the computer-readable media to calculate the score associated with the one or more behaviors based on comparing the biometric data to a standard established by the scenario.

(CRM4) The computer-readable media of any one of (CRM1)-(CRM3), wherein the instructions when executed by the one or more processors, cause the computer-readable media to calculate the score associated with the one or more behaviors based on calculating performance metrics, corresponding to the subject interaction, that indicate how well the subject performs the one or more behaviors in the XR environment.

(CRM5) The computer-readable media of any one of (CRM1)-(CRM4), wherein the instructions, when executed by the one or more processors, cause the computer-readable media to select the second task responsive to use input indicating that the user is unresponsive to the first task, and wherein the instructions, when executed by the one or more processors, cause the computer-readable media to modify the XR environment by presenting the second task without human interaction.

(CRM6) The computer-readable media of any one of (CRM1)-(CRM5), wherein the scenario specifies a task sequence comprising a plurality of different tasks to be performed in an order, and wherein the instructions, when executed by the one or more processors, cause the computer-readable media to select the second task is based on determining a task, of the plurality of different tasks and based on the order, to be performed after the first task.

(CRM7) The computer-readable media of any one of (CRM1)-(CRM6), wherein the instructions, when executed by the one or more processors, cause the computer-readable media to: generate, after modifying the XR environment to present the second task, updated interaction data based on further subject interactions in response to the second task.

(CRM8) The computer-readable media of any one of (CRM1)-(CRM7), wherein the first skill corresponds to one or more of: speech patterns of the subject; eye gaze of the subject; a location of the subject as compared to a location of an avatar object; a decision made in the XR environment; or movement of the subject.

(CRM9) One or more non-transitory computer-readable media comprising instructions that, when executed by at least one processor of an computer-readable media, coupled to an extended reality (XR) device, for assessing Autism Spectrum Disorder (CRMSD), cause the computer-readable media to: receive data identifying one or more behaviors associated with symptoms of ASD for a subject, receive, based on the one or more behaviors, a scenario specifying a plurality of different tasks, wherein the plurality of different tasks are configured to train skills associated with the symptoms of ASD; cause the XR device to present, based on the scenario, an XR environment, cause the XR device to present, in the XR environment and to the subject, a first task of the different tasks specified in the scenario, wherein the first task is configured to train a first skill associated with improvement of at least one of the behaviors associated with the symptoms of ASD, and wherein the first task is configured to prompt the subject to interact with an object in the XR environment; detect one or more interactions with the one or more objects in the XR environment, the one or more interactions being associated with the subject; collect eye tracking data by monitoring, using an eye tracking system of the XR device, eye motions of the subject; generate gaze data by identifying, based on the eye tracking data, one or more second objects in the XR environment which the subject looked at during performance of the task; generate, based on the one or more interactions and based on the gaze data, interaction data that indicates performance, by the subject, of the one or more behaviors associated with the symptoms of ASD; select, from the different tasks and based on the interaction data, a second one of the different tasks, wherein the second task is configured to train a second skill associated with improvement of another one or more of the behaviors associated with the symptoms of ASD, the second skill being different from the first skill; and modify, based on the scenario, the XR environment to present the second task.

(CRM10) The computer-readable media of (CRM9), wherein the instructions, when executed by the one or more processors, cause the computer-readable media to: generate, after modifying the XR environment to present the second task, an updated interaction data based on further subject interactions in response to the second task.

(CRM11) The computer-readable media of (CRM9) or (CRM10), wherein the gaze data indicates whether the subject looked at a particular region of the one or more second objects.

(CRM12) The computer-readable media of (CRM11), wherein the one or more second object comprise an avatar object, and wherein the particular region comprises eyes of the avatar object.

(CRM13) The computer-readable media of any one of (CRM9)-(CRM12), wherein the gaze data indicates whether the subject looked away from the one or more second objects for a period of time.

(CRM14) One or more non-transitory computer-readable media comprising instructions that, when executed by at least one processor of an computer-readable media, coupled to an extended reality (XR) device, for assessing Autism Spectrum Disorder (CRMSD), cause the computer-readable media to receive data identifying one or more behaviors associated with symptoms of ASD for a subject; receive, based on the one or more behaviors, a scenario specifying a plurality of different tasks, wherein the plurality of different tasks are configured to train skills associated with the symptoms of ASD; cause the XR device to present, based on the scenario, an XR environment; cause the XR device to present, in the XR environment and to the subject, a first task of the different tasks specified in the scenario, wherein the first task is configured to train a first skill associated with improvement of at least one of the behaviors associated with the symptoms of ASD, and wherein the first task is configured to prompt the subject to interact with an object in the XR environment, detect one or more interactions with the one or more objects in the XR environment, the one or more interactions being associated with the subject, receive, via one or more microphones of the XR device, voice data corresponding to vocal interaction by the subject, with the one or more object in the XR environment; generate, based on the subject interaction, interaction data that indicates performance by the subject, of the one or more behaviors associated wit the symptoms of ASD: select, from the different tasks and based on the interaction data, a second one of the different tasks, wherein the second task is configured to train a second skill associated with the improvement of another one or more of the behaviors associated with the symptoms of ASD, the second sill being different from the first skill; and modify, based on the scenario, the XR environment to present the second task.

(CRM15) The computer-readable media of (CRM14), wherein the instructions, when executed by the one or more processors, cause the computer-readable media to: generate, after modifying the XR environment to present the second task, updated interaction data based on further subject interactions in response to the second task;

(CRM16) The computer-readable media of (CRM14) or (CRM15), wherein the instructions, when executed by the one or more processors, cause the computer-readable media to generate the interaction data by causing the computer-readable media to calculate, based on the voice data, a confidence score associated with a confidence of the subject when speaking.

(CRM17) The computer-readable media of any one of claims (CRM14)-(CRM16), wherein the instructions, when executed by the one or more processors, cause the computer-readable media it generate the interaction data by causing the computer-readable media to: calculate, based on the voice data, a clarity score associated with a clarity of speech of the subject.

(CRM18) One or more non-transitory computer-readable media comprising instructions that, when executed by at least one processor of an computer-readable media, coupled to an extended reality (XR) device, for assessing Autism Spectrum Disorder (CRMSD), cause the computer-readable media to: receive data identifying one or more behaviors associated with symptoms of ASD for a subject; receive, based on the one or more behaviors, a scenario specifying a plurality of different tasks, wherein the plurality of different tasks are configured to train skills associated with the symptoms of ASD; cause the XR device to present, based on the scenario, an XR environment; cause the XR device to present, in the XR environment and to the subject, a first task of the plurality of different tasks, wherein the first task is configured to train a first skill associated with improvement of at lease one of the behaviors associated with the symptoms of ASD; cause the XR device to present, in the XR environment and to the subject, a second task of the plurality of different tasks, wherein the second task is configured to emulate a daily living skill; collect subject data by monitoring performance, by the subject, of the first task and the second task over a time period; generate, based on the subject interaction, interaction data that indicates performance, by the subject, of the one or more behaviors associated with the symptoms of ASD; select, from a plurality of different tasks and based on the interaction data, a third task configured to train a second skill associated with improvement of at least one of the behaviors associated with the symptoms of ASD; and cause the XR device to modify, based on the template, the XR environment to present the second task and the third task.

(CRM19) The computer-readable media of (CRM18), wherein the instructions, when executed by the one or more processors, cause the computer-readable media to cause the XR device to modify the XR environment by causing the XR environment to present the second task for performance, by the subject in parallel with the third task.

(CRM20) The computer-readable media of (CRM18) or (CRM19), wherein the instructions, when executed by the one or more processors, cause the computer-readable media to cause the XR device to present the second task by causing the XR device to present the second task in parallel with the first task. Although the subject matter has been described in language specific to structural features and/or methodological acts, it is to be understood that the subject matter defined in the appended claims is not necessarily limited to the specific features or acts described above. Rather, the specific features and acts described above are described as example implementations of the following claims.

1. A method for assessing Autism Spectrum Disorder (ASD), the method comprising:
at a computing device that comprises at least one processor and memory and that is coupled to an extended reality (XR) device:
receiving data identifying one or more behaviors associated with symptoms of ASD for a subject;
receiving, based on the one or more behaviors, a scenario specifying a plurality of different tasks, wherein the plurality of different tasks are configured to train skills associated with the symptoms of ASD,
causing the XR device to present, based on the scenario, an XR environment;
causing the XR device to present, in the XR environment and to the subject, a first task of the different tasks specified in the scenario, wherein the first task is configured to train a first skill associated with improvement of at least one of the behaviors associated with the symptoms of ASD, and wherein the first task is configured to prompt the subject to interact with an object in the XR environment;
detecting an interaction with the object in the XR environment, the interaction being associated with the subject:
generating, based on the interaction, interaction data that indicates performance, by the subject, of the one or more behaviors associated with the symptoms of ASD;
selecting, from the different tasks and based on the interaction data, a second one of the different tasks, wherein the second task is configured to train a second skill associated with improvement of another one or more of the behaviors associated with the symptoms of ASD, the second skill being different from the first skill; and
modifying, based on the scenario, the XR environment to present the second task.

2. The method of claim 1, further comprising:
receiving, from one or more biometric tracking devices, biometric data that is associated with the subject and collected during performance of the first task, wherein generating the interaction data comprises generating the interaction data further based on the biometric data.

3. The method of claim 2, wherein calculating the score associated with the one or more behaviors comprises comparing the biometric data to a standard established by the scenario.

4. The method of claim 1, wherein calculating the score associated with the one or more behaviors comprises calculating performance metrics, corresponding to the interaction, that indicate how well the subject performs the one or more behaviors in the XR environment.

5. The method of claim 1, wherein selecting the second task is responsive to the interaction data indicating that the user is unresponsive to the first task, and wherein modifying the XR environment comprises presenting the second task without human interaction.

6. The method of claim 1, wherein the scenario specifies a task sequence comprising a plurality of different tasks to be performed in an order, and wherein selecting the second task is based on determining a task, of the plurality of different tasks and based on the order, to be performed after the first task.

7. The method of claim 1, further comprising:
generating, after modifying the XR environment to present the second task, updated interaction data based on further interactions in response to the second task.

8. The method of claim 1, wherein the first skill corresponds to one or more of:
speech patterns of the subject;
eye gaze of the subject;
a location of the subject as compared to a location of an avatar object;
a decision made in the XR environment; or
movement of the subject.

9. (canceled)

10. The method of claim 29, further comprising:
generating, after modifying the XR environment to present the second task, an updated interaction data based on further interactions in response to the second task.

11. The method of claim 29, wherein the gaze data indicates whether the subject looked at a particular region of the one or more second objects.

12. The method of claim 11, wherein the one or more second objects comprise an avatar object, and wherein the particular region comprises eyes of the avatar object.

13. The method of claim 29, wherein the gaze data indicates whether the subject looked away from the one or more second objects for a period of time.

14.-20. (canceled)

21. An apparatus, coupled to an extended reality (XR) device, for assessing Autism Spectrum Disorder (ASD), the apparatus comprising:
one or more processors; and
memory storing instructions that, when executed by the one or more processors, cause the apparatus to:
receive data identifying one or more behaviors associated with symptoms of ASD for a subject;
receive, based on the one or more behaviors, a scenario specifying a plurality of different tasks, wherein the plurality of different tasks are configured to train skills associated with the symptoms of ASD;
cause the XR device to present, based on the scenario, an XR environment;
cause the XR device to present, in the XR environment and to the subject, a first task of the different tasks specified in the scenario, wherein the first task is configured to train a first skill associated with improvement of at least one of the behaviors associated with the symptoms of ASD, and wherein the first task is configured to prompt the subject to interact with an object in the XR environment;
detect an interaction with the object in the XR environment;
generate, based on the interaction, interaction data that indicates performance, by the subject, of the one or more behaviors associated with the symptoms of ASD;
select, from the different tasks and based on the interaction data, a second one of the different tasks, wherein the second task is configured to train a second skill associated with improvement of another one or more of the behaviors associated with the symptoms of ASD, the second skill being different from the first skill; and
modify, based on the scenario, the XR environment to present the second task.

22.-24. (canceled)

25. One or more non-transitory computer-readable media comprising instructions that, when executed by at least one processor of an apparatus, coupled to an extended reality (XR) device, for assessing Autism Spectrum Disorder (ASD), cause the apparatus to:
receive data identifying one or more behaviors associated with symptoms of ASD for a subject;
receive, based on the one or more behaviors, a scenario specifying a plurality of different tasks, wherein the plurality of different tasks are configured to train skills associated with the symptoms of ASD;
cause the XR device to present, based on the scenario, an XR environment;
cause the XR device to present, in the XR environment and to the subject, a first task of the different tasks specified in the scenario, wherein the first task is configured to train a first skill associated with improvement of at least one of the behaviors associated with the symptoms of ASD, and wherein the first task is configured to prompt the subject to interact with an object in the XR environment;
detect an interaction with the object in the XR environment;
generate, based on the interaction, interaction data that indicates performance, by the subject, of the one or more behaviors associated with the symptoms of ASD;
select, from the different tasks and based on the interaction data, a second one of the different tasks, wherein the second task is configured to train a second skill associated with improvement of another one or more of the behaviors associated with the symptoms of ASD, the second skill being different from the first skill; and
modify, based on the scenario, the XR environment to present the second task.

26.-28. (canceled)

29. The method of claim 1, further comprising:
collecting eye tracking data by monitoring, using an eye tracking system of the XR device, eye motions of the subject; and
generating gaze data by identifying, based on the eye tracking data, one or more second objects in the XR environment which the subject looked at during performance of the task;
wherein generating interaction data is further based on the gaze data.

30. The method of claim 1, further comprising:
receiving, via one or more microphones of the XR device, voice data corresponding to vocal interaction, by the subject, with the one or more objects in the environment; and
either calculating, based on the voice data, a confidence score associated with a confidence of the subject when speaking;
or calculating, based on the voice data, a clarity score associated with a clarity of speech of the subject.
